(19) United States
(12) Patent Application Publication
(10) Pub. No.: US 2023/0095750 A1
(43) Pub. Date: Mar. 30, 2023

(54) FLOW CHEMISTRY SYNTHESIS OF ISOCYANATES

(71) Applicant: The Regents of the University of California, Oakland, CA (US)

(72) Inventors: Michael D. Burkart, La Jolla, CA (US); Thien An Phung Hai, La Jolla, CA (US)

(21) Appl. No.: 17/783,511

(22) PCT Filed: Dec. 14, 2020
(86) PCT No.: PCT/US2020/064898

§ 371 Date: Jun. 8, 2022

Related U.S. Application Data
(60) Provisional application No. 62/947,345, filed on Dec. 12, 2019.

(51) Int. Cl. C07C 263/12; C07C 243/14; C07C 247/02

(52) U.S. Cl. CPC C07C 263/12; C07C 243/14; C07C 247/02

(57) ABSTRACT

The disclosure provides, inter alia, safe and environmentally-friendly methods, such as flow chemistry, to synthesize isocyanates, such as methylene diphenyl diisocyanate, toluene diisocyanate, hexamethylene diisocyanate, isophorone diisocyanate, and tetramethylxylene diisocyanate.

### CROSS-REFERENCE TO RELATED APPLICATIONS

This application claims priority to U.S. Application No. 62/947,345 filed Dec. 12, 2019, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under grant number DOE-PEAK-21C42A awarded by the U.S. Department of Energy. The government has certain rights in the invention.

### BACKGROUND

Isocyanates serve as important and versatile chemical intermediates in the manufacture of diverse products, from flexible and rigid polyurethane foams, to agrochemicals and pharmaceuticals. (Refs 1-3). The production of isocyanates draws mainly from petrochemical raw materials, including benzene, toluene, propylene and aniline, and they are produced industrially using phosgenation of alkyl or aromatic amines. (Refs 2, 4). While other preparative strategies have been proposed, none have replaced standard methodologies. (Refs 5, 6). Of these reported methods, the Curtius rearrangement is considered one of the cleanest and highest-yielding approaches for isocyanate synthesis; the reagents are relatively inexpensive, and the byproducts are inert. However, safety concerns around the preparation of intermediate acyl azides have barred this method from reaching multi-kilogram scale production. (Ref 4). Thus, there is a need in the art for processes that have improved reaction safety, accelerated reaction kinetics, clean production methods, and improved scale-up potential. The disclosure is directed to these, as well as other, important ends.

### BRIEF SUMMARY

The disclosure provides a safe and environmentally-friendly continuous flow process for producing an isocyanate by: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) removing water from the second solution in flow to produce a third solution comprising the azide compound and the organic solvent; and (iv) heating the third solution comprising the azide compound in flow to produce the isocyanate. The isocyanate can be a diisocyanate or a monoisocyanate. The diisocyanate can be methylene diphenyl diisocyanate, toluene diisocyanate, hexamethylene diisocyanate, isophorone diisocyanate, or tetramethylxylene diisocyanate.

These and other embodiments of the disclosure are described in more detail herein.

### BRIEF DESCRIPTION OF THE DRAWINGS

FIG. 1 illustrates a flow chemistry diagram for synthesis of isocyanates from acyl hydrazides.

FIG. 2 illustrates a flow chart for synthesis of heptamethylene diisocyanate.

FIG. 3 illustrates isolation of azelaic acid from algae oil.

FIG. 4 illustrates a set up flow system to synthesize isocyanates.

### DETAILED DESCRIPTION

### Definitions

The abbreviations used herein have their conventional meaning within the chemical arts. The chemical structures and formulae set forth herein are constructed according to the standard rules of chemical valency known in the chemical arts.

The term “continuous flow process” or “flow chemistry” refers to a chemical reaction that is run in a continuously flowing stream rather than in batch production. For a continuous flow process, multiple reactor and separation modules can be arranged consecutively to form a single continuous operation unit. For continuous multistep synthesis, reagents are fed into the assembly at specified points and the reaction stream is manipulated along the reaction path to suit the needs of individual transformations. Reaction intermediates are thus generated and directly consumed in the closed environment of the reactor.

The term “in flow” refers to a process step or reaction that is conducted in a continuous flow process.

The term “acyl hydrazide” refers to a compound having the moiety —C(O)NHNH₂.

The term “acyl azide” refers to a compound having the moiety —C(O)N₃.

The term “isocyanate” refers to a compound having the moiety —N═C═O (alternatively written as —NCO). A “diisocyanate” refers to a compound having two isocyanate moieties. A “monoisocyanate” refers to a compound having one isocyanate moiety.

The term “nitrous acid” refers to NHO₂. Nitrous acid can be made by any method known in the art. Nitrous acid can be made by reacting sodium nitrite (NaNO₂) with a mineral acid (e.g., HCl, HBr). Nitrous acid can be made by dissolving dinitrogen trioxide (N₂O₃) in water. In embodiments, the nitrous acid is made from sodium nitrite (NaNO₂) and HCl.

The term “Curtius rearrangement” refers to the thermal decomposition of an acyl azide to an isocyanate with loss of nitrogen gas.

The term “solution” is used in accordance with its ordinary meaning and refers to a liquid mixture in which one component (e.g., a solute or compound) is uniformly distributed within another component (e.g., a solvent).

The term “organic solvent” as used herein is used in accordance with its ordinary meaning in chemistry and refers to a solvent which includes carbon.

The term “methylene diphenyl diisocyanate” or “MDI” is represented by the structure:

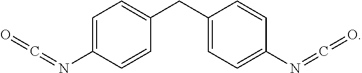 [SMILES: O=C=Nc1ccc(Cc2ccc(N=C=O)cc2)cc1]

The term “toluene diisocyanate” is represented by the structure:

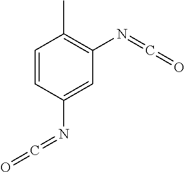

The term “hexamethylene diisocyanate” is represented by the structure:

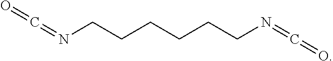 [SMILES: O=C=NCCCCCCN=C=O]

The term “isophorone diisocyanate” is represented by the structure:

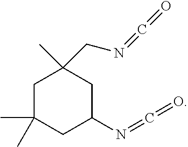 [SMILES: CC1(C)CC(N=C=O)CC(C)(CN=C=O)C1]

The term “tetramethylxylene diisocyanate” is represented by the structure:

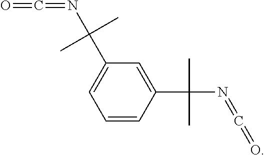 [SMILES: CC(C)(N=C=O)c1cccc(C(C)(C)N=C=O)c1]

The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ to means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]-heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl.

The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydro-benzofuran-2-yl, 2,3-dihydrobenzofuran-3-yl, indolin-1-yl, indolin-2-yl, indolin-3-yl, 2,3-dihydro-benzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” and “-” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

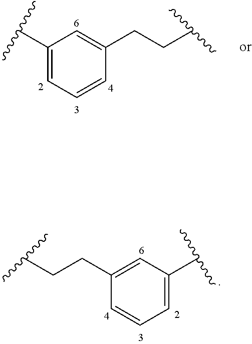 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is selected as are each R′, R″, R′″, and R″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NW—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈alkyl, C₁-C₆alkyl, or C₁-C₄alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈cycloalkyl, C₃-C₆cycloalkyl, or C₅-C₆cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

A “substituent group,” as used herein, means a group selected from the following moieties: (B) alkyl (e.g., C₁-C₈alkyl, C₁-C₆alkyl, or C₁-C₄alkyl), heteroalkyl (e.g., 2 to 8 membered heteroalkyl, 2 to 6 membered heteroalkyl, or 2 to 4 membered heteroalkyl), cycloalkyl (e.g., C₃-C₈cycloalkyl, C₃-C₆cycloalkyl, or C₅-C₆cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: (i) oxo, halogen, —CCl₃, —CF₃, —Cl₃, —CHCl₂, —CHBr₂, —CHF₂, —CH₂Cl, —CH₂Br, —CH₂F, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHF₂, —OCH₂Cl, —OCH₂Br, —OCH₂F, —N₃, unsubstituted alkyl (e.g., C₁-C₈ alkyl, C₁-C₆alkyl, or C₁-C₄alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈cycloalkyl, C₃-C₆cycloalkyl, or C₅-C₆cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl (e.g., C₁-C₈alkyl, C₁-C₆alkyl, or C₁-C₄alkyl), heteroalkyl (e.g., 2 to 8 membered heteroalkyl, 2 to 6 membered heteroalkyl, or 2 to 4 membered heteroalkyl), cycloalkyl (e.g., C₃-C₈cycloalkyl, C₃-C₆cycloalkyl, or C₅-C₆cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: (a) oxo, halogen, —CCl₃, —CF₃, —CI₃, —CHCl₂, —CHBr₂, —CHF₂, —CH₂Cl, —CH₂Br, —CH₂F, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHF₂, —OCH₂Cl, —OCH₂Br, —OCH₂F, —N₃, unsubstituted alkyl (e.g., C₁-C₈alkyl, C₁-C₆alkyl, or C₁-C₄alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈cycloalkyl, C₃-C₆cycloalkyl, or C₅-C₆cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl (e.g., C₁-C₈alkyl, C₁-C₆alkyl, or C₁-C₄alkyl), heteroalkyl (e.g., 2 to 8 membered heteroalkyl, 2 to 6 membered heteroalkyl, or 2 to 4 membered heteroalkyl), cycloalkyl (e.g., C₃-C₈cycloalkyl, C₃-C₆cycloalkyl, or C₅-C₆cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈alkyl, C₁-C₆alkyl, or C₁-C₄alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈cycloalkyl, C₃-C₆cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In embodiments, each substituted group described in the compounds herein is substituted with at least one substituent group. In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments, each substituted or unsubstituted alkyl may be a substituted or unsubstituted C₁-C₂₀alkyl, each substituted or unsubstituted heteroalkyl is a substituted or unsubstituted 2 to 20 membered heteroalkyl, each substituted or unsubstituted cycloalkyl is a substituted or unsubstituted C₃-C₈cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In embodiments, the compound (e.g., nucleotide analogue) is a chemical species set forth in the Examples section, claims, embodiments, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group is optionally different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group is optionally different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group is optionally different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the disclosure do not include those that are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀alkyl, or unsubstituted 2 to 20 membered heteroalkyl,” the group may contain one or more unsubstituted C₁-C₂₀alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

Methods

Diisocyanates used in polyurethanes are commonly prepared by phosgenation of petroleum-sourced diamines. This involves highly toxic phosgene and produces corrosive HCl, limiting synthetic applications. In our search for a renewable source for diisocyanates, the inventors developed a practical methodology for the production of isocyanates from algae biomass-derived fatty acids or other renewable sources. This technique utilizes flow chemistry to prepare and convert high energy intermediates, thus mitigating safety concerns. Using continuous flow, acyl azides are prepared from acyl hydrazides and subsequently heated to undergo Curtius rearrangement, affording isocyanates in one scalable process. The method is efficient, safe, and sustainable and offers an opportunity to prepare isocyanates and diisocyanates from renewable feedstocks and amenable to distributed manufacturing processes.

The disclosure provides a process for producing at least one gram of an isocyanate comprising: (i) mixing an acyl hydrazide with an aqueous solution comprising nitrous acid to form a first solution comprising an acyl azide and water; (ii) mixing an organic solvent with the first solution to produce a second solution comprising the acyl azide compound, the organic solvent, and water; and (iii) heating the third solution comprising the azide compound to produce the isocyanate. In embodiments, the process is for producing a least one kilogram of an isocyanate. In embodiments, the process is for producing a least five-hundred kilograms of an isocyanate. In embodiments, the process is for producing a least a metric ton of an isocyanate. In embodiments, the process is a continuous flow process.

The disclosure provides a process for producing at least one gram of an isocyanate comprising: (i) mixing an acyl hydrazide with an aqueous solution comprising nitrous acid to form a first solution comprising an acyl azide and water; (ii) mixing an organic solvent with the first solution to produce a second solution comprising the acyl azide compound, the organic solvent, and water; (iii) removing water from the second solution to produce a third solution comprising the azide compound and the organic solvent; and (iv) heating the third solution comprising the azide compound to produce the isocyanate. In embodiments, the method further comprises drying the third solution in flow prior to heating the third solution, where drying the third solution removes residual water from the organic solvent prior to heating the third solution to perform the Curtius rearrangement. In embodiments, the process is for producing a least one kilogram of an isocyanate. In embodiments, the process is for producing a least five-hundred kilograms of an isocyanate. In embodiments, the process is for producing a least a metric ton of an isocyanate. In embodiments, the process is a continuous flow process.

The disclosure provides a process for producing at least one gram of an isocyanate comprising: (i) mixing an acyl hydrazide with an aqueous solution comprising nitrous acid to form a first solution comprising an acyl azide and water; (ii) mixing an organic solvent with the first solution to produce a second solution comprising the acyl azide compound, the organic solvent, and water; (iii) removing water from the second solution to produce a third solution comprising the azide compound and the organic solvent; (iv) drying the third solution prior; and (v) heating the third solution comprising the azide compound in flow to produce the isocyanate. In embodiments, the process is for producing a least one kilogram of an isocyanate. In embodiments, the process is for producing a least five-hundred kilograms of an isocyanate. In embodiments, the process is for producing a least a metric ton of an isocyanate. In embodiments, the process is a continuous flow process.

The disclosure provides a continuous flow process for producing an isocyanate comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; and (iii) heating the third solution comprising the azide compound in flow to produce the isocyanate. The isocyanate can be a monoisocyanate or a diisocyanate.

The disclosure provides a continuous flow process for producing an isocyanate comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) removing water from the second solution in flow to produce a third solution comprising the azide compound and the organic solvent; and (iv) heating the third solution comprising the azide compound in flow to produce the isocyanate. In embodiments, the method further comprises drying the third solution in flow prior to heating the third solution, where drying the third solution removes residual water from the organic solvent prior to heating the third solution to perform the Curtius rearrangement. The isocyanate can be a monoisocyanate or a diisocyanate.

The disclosure provides a continuous flow process for producing an isocyanate comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) removing water from the second solution in flow to produce a third solution comprising the azide compound and the organic solvent; (iv) drying the third solution in flow; and (v) heating the third solution comprising the azide compound in flow to produce the isocyanate. The isocyanate can be a monoisocyanate or a diisocyanate.

In embodiments of the methods described herein, the organic solvent comprises acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, dimethyl ether, 1,2-dimethoxyethane, dimethylformamide, dimethyl sulfoxide, 1,4-dioxane, ethanol, ethyl acetate, ethylene glycol, glycerin, heptane, hexamethyl-phosphoramide, hexamethylphosphorous, triamide, hexane, methanol, methyl t-butyl ether, methylene chloride, N-methyl-2-pyrrolidinone, nitromethane, pentane, petroleum ether, 1-propanol, 2-propanol, pyridine, tetrahydrofuran, toluene, triethyl amine, o-xylene, m-xylene, p-xylene, or a mixture of two or more of the foregoing. In embodiments, the organic solvent comprises chloroform, toluene, benzene, methyl tert-butyl ether, tetrahydrofuran, or a mixture of two or more thereof. In embodiments, the organic solvent comprises chloroform. In embodiments, the organic solvent comprises toluene. In embodiments, the organic solvent comprises benzene. In embodiments, the organic solvent comprises methyl tert-butyl ether. In embodiments, the organic solvent comprises tetrahydrofuran.

In embodiments of the methods described herein, the process comprises mixing the acyl hydrazide with an aqueous solution comprising nitrous acid in order to convert the acyl hydrazide into an acyl azide. This reaction can be performed at a temperature from about 0° C. to about 60° C. In embodiments, the reaction is performed at a temperature from about 0° C. to about 30° C. In embodiments, the reaction is performed at a temperature from about 0° C. to about 25° C. In embodiments, the reaction is performed at a temperature from about 15° C. to about 30° C. In embodiments, the reaction is performed at a temperature from about 20° C. to about 25° C. In embodiments, the reaction is performed at a temperature from about 20° C. to about 22° C.

In embodiments of the methods described herein, the process comprises heating the third solution to a temperature from about 65° C. to about 120° C. in order to convert the acyl azide into an isocyanate by Curtius rearrangement. In embodiments, the process comprises heating the third solution at a temperature from about 70° C. to about 110° C. In embodiments, the process comprises heating the third solution at a temperature from about 70° C. to about 105° C. In embodiments, the process comprises heating the third solution at a temperature from about 75° C. to about 100° C. In embodiments, the process comprises heating the third solution at a temperature from about 80° C. to about 100° C. In embodiments, the process comprises heating the third solution at a temperature from about 85° C. to about 100° C. In embodiments, the process comprises heating the third solution at a temperature from about 80° C. to about 95° C. In embodiments, the process comprises heating the third solution at a temperature from about 80° C. to about 90° C. In embodiments, the process comprises heating the third solution at a temperature of about 75° C. In embodiments, the process comprises heating the third solution at a temperature of about 80° C. In embodiments, the process comprises heating the third solution at a temperature of about 85° C. In embodiments, the process comprises heating the third solution at a temperature of about 90° C. In embodiments, the process comprises heating the third solution at a temperature of about 95° C. In embodiments, the process comprises heating the third solution at a temperature of about 100° C. The skilled artisan will appreciate that the temperature required to perform the Curtius rearrangement will be dependent upon the acyl azide that is being converted into an isocyanate. For example, aromatic azyl azides generally require a higher temperature to perform the Curtius rearrangement than aliphatic azyl azides. In embodiments, the methods comprise heating the third solution containing an aliphatic azyl azide at a temperature from about 80° C. to about 95° C. In embodiments, the methods comprise heating the third solution containing an aliphatic azyl azide at a temperature from about 85° C. to about 90° C. In embodiments, the methods comprise heating the third solution containing an aromatic azyl azide at a temperature from about 90° C. to about 105° C. In embodiments, the methods comprise heating the third solution containing an aromatic azyl azide at a temperature from about 95° C. to about 100° C.

In embodiments of the methods described herein, the acyl hydrazide is mixed with an aqueous solution comprising nitrous acid in amounts effective to convert the acyl hydrazide to an acyl azide. In embodiments, the ratio of acyl hydrazide to nitrous acid is from about 1:1 to about 1:3. In embodiments, the ratio of acyl hydrazide to nitrous acid is about 1:2.

In embodiments of the methods described herein, the organic solvent is mixed with the first solution comprising the acyl azide in amounts effective for the reaction process. In embodiments, the volume of organic solvent to the volume of the first solution comprising the acyl azide is from about 2:1 to about 1:2. In embodiments, the volume of organic solvent to the volume of the first solution comprising the acyl azide is about 1:1.

In embodiments of the methods described herein, the flow rate for the continuous flow process can be any flow rate necessary to produce the isocyanates described herein. In embodiments, the flow rate can remain constant throughout the process. In embodiments, the flow rate can be varied at any point in the process. In embodiments, the flow rate is at least 1 mL min⁻¹. In embodiments, the flow rate is at least 10 mL min⁻¹. In embodiments, the flow rate is at least 25 mL min⁻¹. In embodiments, the flow rate is at least 50 mL min⁻¹. In embodiments, the flow rate is at least 75 mL min⁻¹. In embodiments, the flow rate is at least 100 mL min⁻¹.

In embodiments of the methods described herein, the continuous flow process produces isocyanates at rate of at least 1 g h⁻¹. In embodiments, the continuous flow process produces isocyanates at rate of at least 5 g h⁻¹. In embodiments, the continuous flow process produces isocyanates at rate of at least 10 g h⁻¹. In embodiments, the continuous flow process produces isocyanates at rate of at least 25 g h⁻¹. In embodiments, the continuous flow process produces isocyanates at rate of at least 50 g h⁻¹. In embodiments, the continuous flow process produces isocyanates at rate of at least 100 g h⁻¹. In embodiments, the continuous flow process produces isocyanates at rate of at least 1 kg h⁻¹.

In embodiments, the method comprises drying the third solution in flow prior to heating the third solution in order to remove residual water from the organic solvent prior to performing the Curtius rearrangement. The organic solvent can be dried by any method known in the art. In embodiments, the organic solvent is dried by storage with molecular sieves or by passage through molecular sieves (e.g., 3 Å or 4 Å molecular sieves). In embodiments, the organic solvent is dried with any drying agent know in the art, such as an anhydrous inorganic salt. Such anhydrous inorganic salts include calcium chloride, calcium sulfate, potassium carbonate, and sodium sulfate.

Compounds

In embodiments, the acyl hydrazide has the formula:

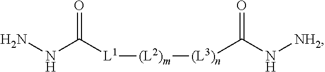

where m, n, L¹, L², and L³ are as defined herein.

In embodiments, the acyl azide has the formula:

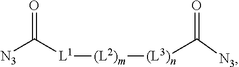

where m, n, L¹, L², and L³ are as defined herein.

In embodiments, the isocyanate is a diisocyanate having the formula:

O═C═N-L¹-(L²)ₘ-(L³)ₙ-N═C═O,

where m, n, L¹, L², and L³ are as defined herein.

m and n are independently 0 or 1. In embodiments, m and n are 0. In embodiments, m is 1 and n is 0. In embodiments, m and n are 1.

L¹, L², and L³ are each independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹, L², and L³ are each independently substituted or unsubstituted alkylene, substituted or unsubstituted cycloalkylene, substituted or unsubstituted arylene, or substituted or unsubstituted heteroarylene. In embodiments, L¹, L², and L³ are each independently substituted or unsubstituted C₁-C₁₂alkylene, substituted or unsubstituted C₅-C₆cycloalkylene, substituted or unsubstituted C₅-C₆ arylene, or substituted or unsubstituted 5 or 6 membered heteroarylene. In embodiments, L¹ is substituted or unsubstituted C₁-C₁₂alkylene. In embodiments, L¹ is substituted or unsubstituted C₅-C₆cycloalkylene. In embodiments, L¹ is substituted or unsubstituted C₅-C₆ arylene. In embodiments, L¹ is substituted or unsubstituted 5 or 6 membered heteroarylene. In embodiments, L² is substituted or unsubstituted C₁-C₁₂alkylene. In embodiments, L² is substituted or unsubstituted C₅-C₆ cycloalkylene. In embodiments, L² is substituted or unsubstituted C₅-C₆ arylene. In embodiments, L² is substituted or unsubstituted 5 or 6 membered heteroarylene. In embodiments, L³ is substituted or unsubstituted C₁-C₁₂alkylene. In embodiments, L³ is substituted or unsubstituted C₅-C₆cycloalkylene. In embodiments, L³ is substituted or unsubstituted C₅-C₆ arylene. In embodiments, L³ is substituted or unsubstituted 5 or 6 membered heteroarylene.

In embodiments, L¹ is L¹¹-substituted or unsubstituted alkylene (e.g., C₁-C₈, C₁-C₆, or C₁-C₄), L¹¹-substituted or unsubstituted heteroalkylene (e.g., 2 to 10 membered, 2 to 8 membered, 4 to 8 membered, 2 to 6 membered, or 2 to 4 membered), L¹¹-substituted or unsubstituted cycloalkylene (e.g., C₃-C₈, C₄-C₈, or C₅-C₆), L¹¹-substituted or unsubstituted heterocycloalkylene (e.g., 3 to 8 membered, 4 to 8 membered, or 5 to 6 membered), L¹¹-substituted or unsubstituted arylene (e.g., C₅-C₁₀ or C₅-C₆), or L¹¹-substituted or unsubstituted heteroarylene (e.g., 5 to 10 membered, 5 to 9 membered, or 5 to 6 membered). L¹¹ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, L¹¹-substituted or unsubstituted alkyl (e.g., C₁-C₈, C₁-C₆, or C₁-C₄), L¹¹-substituted or unsubstituted heteroalkyl (e.g., 2 to 10 membered, 2 to 8 membered, 4 to 8 membered, 2 to 6 membered, or 2 to 4 membered), L¹¹-substituted or unsubstituted cycloalkyl (e.g., C₃-C₈, C₄-C₈, or C₅-C₆), L¹¹-substituted or unsubstituted heterocycloalkyl (e.g., 3 to 8 membered, 4 to 8 membered, or 5 to 6 membered), L¹¹-substituted or unsubstituted aryl (e.g., C₅-C₁₀ or C₅-C₆), or L¹¹-substituted or unsubstituted heteroaryl (e.g., 5 to 10 membered, 5 to 9 membered, or 5 to 6 membered).

L¹¹ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl (e.g., C₁-C₈, C₁-C₆, or C₁-C₄), unsubstituted heteroalkyl (e.g., 2 to 10 membered, 2 to 8 membered, 4 to 8 membered, 2 to 6 membered, or 2 to 4 membered), unsubstituted cycloalkyl (e.g., C₃-C₈, C₄-C₈, or C₅-C₆), unsubstituted heterocycloalkyl (e.g., 3 to 8 membered, 4 to 8 membered, or 5 to 6 membered), unsubstituted aryl (e.g., C₆-C₁₀ or C₆), or unsubstituted heteroaryl (e.g., 5 to 10 membered, 5 to 9 membered, or 5 to 6 membered).

In embodiments, L¹ is unsubstituted C₁-C₁₂alkylene. In embodiments, L¹ is unsubstituted C₅-C₆cycloalkylene. In embodiments, L¹ is unsubstituted C₅-C₆ arylene. In embodiments, L¹ is unsubstituted 5 or 6 membered heteroarylene. In embodiments, L¹ is substituted C₁-C₁₂alkylene, wherein the substituent is a C₁₋₄alkyl. In embodiments, L¹ is substituted C₅-C₆cycloalkylene, wherein the substituent is a C₁₋₄alkyl. In embodiments, L¹ is substituted C₅-C₆ arylene, wherein the substituent is a C₁₋₄alkyl. In embodiments, L¹ is substituted 5 or 6 membered heteroarylene, wherein the substituent is a C₁₋₄alkyl. In embodiments, L¹ has one C₁₋₄alkyl substituent. In embodiments, L¹ has two C₁₋₄alkyl substituents. In embodiments, L¹ has three C₁₋₄alkyl substituent.

In embodiments, L¹ is —CH₂—, m is 0, and n is 0. In embodiments, L¹ is —(CH₂)₂—, m is 0, and n is 0. In embodiments, L¹ is —(CH₂)₃—, m is 0, and n is 0. In embodiments, L¹ is —(CH₂)₄—, m is 0, and n is 0. In embodiments, L¹ is —(CH₂)₅—, m is 0, and n is 0. In embodiments, L¹ is —(CH₂)₆—, m is 0, and n is 0. In embodiments, L¹ is —(CH₂)₇—, m is 0, and n is 0. In embodiments, L¹ is —(CH₂)₈—, m is 0, and n is 0. In embodiments, L¹ is —(CH₂)₉—, m is 0, and n is 0. In embodiments, L¹ is —(CH₂)₁₀—, m is 0, and n is 0. In embodiments, L¹ is

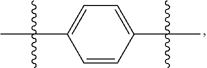 [SMILES: *c1ccc(*)cc1]

m is 0, and n is 0. In embodiments, L¹ is

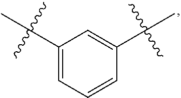 [SMILES: *c1cccc(*)c1]

m is 0, and n is 0. In embodiments, L¹ is

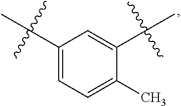 [SMILES: *c1ccc(C)c(*)c1]

m is 0, and n is 0. In embodiments, L¹ is

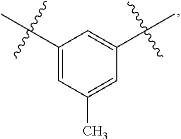 [SMILES: *c1cc(*)cc(C)c1]

m is 0, and n is 0. In embodiments, L¹ is

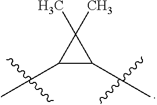 [SMILES: *C1C(*)C1(C)C]

In embodiments, L¹ is

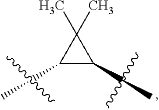 [SMILES: *[C@H]1[C@H](*)C1(C)C]

m is 0, and n is 0.

In embodiments, L¹ is

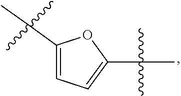 [SMILES: *c1ccc(*)o1]

m is 0, and n is 0.

In embodiments, L² is L²²-substituted or unsubstituted alkylene (e.g., C₁-C₈, C₁-C₆, or C₁-C₄), L²²-substituted or unsubstituted heteroalkylene (e.g., 2 to 10 membered, 2 to 8 membered, 4 to 8 membered, 2 to 6 membered, or 2 to 4 membered), L²²-substituted or unsubstituted cycloalkylene (e.g., C₃-C₈, C₄-C₈, or C₅-C₆), L²²-substituted or unsubstituted heterocycloalkylene (e.g., 3 to 8 membered, 4 to 8 membered, or 5 to 6 membered), L²²-substituted or unsubstituted arylene (e.g., C₅-C₁₀ or C₅-C₆), or L²²-substituted or unsubstituted heteroarylene (e.g., 5 to 10 membered, 5 to 9 membered, or 5 to 6 membered).

L²² is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, L²²²-substituted or unsubstituted alkyl (e.g., C₁-C₈, C₁-C₆, or C₁-C₄), L²²²-substituted or unsubstituted heteroalkyl (e.g., 2 to 10 membered, 2 to 8 membered, 4 to 8 membered, 2 to 6 membered, or 2 to 4 membered), L²²²-substituted or unsubstituted cycloalkyl (e.g., C₃-C₈, C₄-C₈, or C₅-C₆), L²²²-substituted or unsubstituted heterocycloalkyl (e.g., 3 to 8 membered, 4 to 8 membered, or 5 to 6 membered), L²²²-substituted or unsubstituted aryl (e.g., C₅-C₁₀ or C₅-C₆), or L²²²-substituted or unsubstituted heteroaryl (e.g., 5 to 10 membered, 5 to 9 membered, or 5 to 6 membered).

L²²² is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl (e.g., C₁-C₈, C₁-C₆, or C₁-C₄), unsubstituted heteroalkyl (e.g., 2 to 10 membered, 2 to 8 membered, 4 to 8 membered, 2 to 6 membered, or 2 to 4 membered), unsubstituted cycloalkyl (e.g., C₃-C₈, C₄-C₈, or C₅-C₆), unsubstituted heterocycloalkyl (e.g., 3 to 8 membered, 4 to 8 membered, or 5 to 6 membered), unsubstituted aryl (e.g., C₆-C₁₀ or C₆), or unsubstituted heteroaryl (e.g., 5 to 10 membered, 5 to 9 membered, or 5 to 6 membered).

In embodiments, L² is unsubstituted C₁-C₁₂alkylene. In embodiments, L² is unsubstituted C₅-C₆cycloalkylene. In embodiments, L² is unsubstituted C₅-C₆ arylene. In embodiments, L² is unsubstituted 5 or 6 membered heteroarylene. In embodiments, L² is substituted C₁-C₁₂alkylene, wherein the substituent is a C₁₋₄alkyl. In embodiments, L² is substituted C₅-C₆cycloalkylene, wherein the substituent is a C₁₋₄alkyl. In embodiments, L² is substituted C₅-C₆ arylene, wherein the substituent is a C₁₋₄alkyl. In embodiments, L² is substituted 5 or 6 membered heteroarylene, wherein the substituent is a C₁₋₄alkyl. In embodiments, L² has one C₁₋₄alkyl substituent. In embodiments, L² has two C₁₋₄alkyl substituents. In embodiments, L² has three C₁-4 alkyl substituent.

In embodiments, L¹ is methylene, m is 1 and L² is

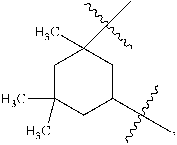 [SMILES: *C1CC(*)(C)CC(C)(C)C1]

and n is 0.

In embodiments, L³ is L³³-substituted or unsubstituted alkylene (e.g., C₁-C₈, C₁-C₆, or C₁-C₄), L³³-substituted or unsubstituted heteroalkylene (e.g., 2 to 10 membered, 2 to 8 membered, 4 to 8 membered, 2 to 6 membered, or 2 to 4 membered), L³³-substituted or unsubstituted cycloalkylene (e.g., C₃-C₈, C₄-C₈, or C₅-C₆), L³³-substituted or unsubstituted heterocycloalkylene (e.g., 3 to 8 membered, 4 to 8 membered, or 5 to 6 membered), L³³-substituted or unsubstituted arylene (e.g., C₅-C₁₀ or C₅-C₆), or L³³-substituted or unsubstituted heteroarylene (e.g., 5 to 10 membered, 5 to 9 membered, or 5 to 6 membered).

L³³ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, L³³³-substituted or unsubstituted alkyl (e.g., C₁-C₈, C₁-C₆, or C₁-C₄), L³³³-substituted or unsubstituted heteroalkyl (e.g., 2 to 10 membered, 2 to 8 membered, 4 to 8 membered, 2 to 6 membered, or 2 to 4 membered), L³³³-substituted or unsubstituted cycloalkyl (e.g., C₃-C₈, C₄-C₈, or C₅-C₆), L³³³-substituted or unsubstituted heterocycloalkyl (e.g., 3 to 8 membered, 4 to 8 membered, or 5 to 6 membered), L³³³-substituted or unsubstituted aryl (e.g., C₅-C₁₀ or C₅-C₆), or L³³³-substituted or unsubstituted heteroaryl (e.g., 5 to 10 membered, 5 to 9 membered, or 5 to 6 membered).

L³³³ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl (e.g., C₁-C₈, C₁-C₆, or C₁-C₄), unsubstituted heteroalkyl (e.g., 2 to 10 membered, 2 to 8 membered, 4 to 8 membered, 2 to 6 membered, or 2 to 4 membered), unsubstituted cycloalkyl (e.g., C₃-C₈, C₄-C₈, or C₅-C₆), unsubstituted heterocycloalkyl (e.g., 3 to 8 membered, 4 to 8 membered, or 5 to 6 membered), unsubstituted aryl (e.g., C₆-C₁₀ or C₆), or unsubstituted heteroaryl (e.g., 5 to 10 membered, 5 to 9 membered, or 5 to 6 membered).

In embodiments, L³ is unsubstituted C₁-C₁₂alkylene. In embodiments, L³ is unsubstituted C₅-C₆cycloalkylene. In embodiments, L³ is unsubstituted C₅-C₆ arylene. In embodiments, L³ is unsubstituted 5 or 6 membered heteroarylene. In embodiments, L³ is substituted C₁-C₁₂alkylene, wherein the substituent is a C₁-4 alkyl. In embodiments, L³ is substituted C₅-C₆cycloalkylene, wherein the substituent is a C₁-4 alkyl. In embodiments, L³ is substituted C₅-C₆ arylene, wherein the substituent is a C₁-4 alkyl. In embodiments, L³ is substituted 5 or 6 membered heteroarylene, wherein the substituent is a C₁₋₄alkyl. In embodiments, L³ has one C₁-4 alkyl substituent. In embodiments, L³ has two C₁-4 alkyl substituents. In embodiments, L³ has three C₁-4 alkyl substituent.

In embodiments, L¹ is

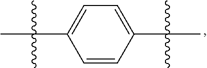 [SMILES: *c1ccc(*)cc1]

m is 1 and L² is methylene, and n is 1 and L³ is

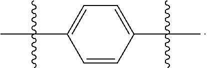 [SMILES: *c1ccc(*)cc1]

In embodiments, L¹ is

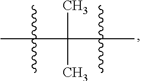 [SMILES: *C(*)(C)C]

### m is 1 and L² is

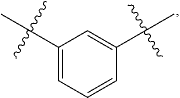 [SMILES: *c1cccc(*)c1]

### and n is 1 and L³ is

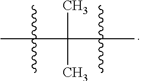 [SMILES: *C(*)(C)C]

In embodiments, the diisocyanate is methylene diphenyl diisocyanate, toluene diisocyanate, hexamethylene diisocyanate, isophorone diisocyanate, tetramethylxylene diisocyanate, or a combination of two or more thereof. In embodiments, the diisocyanate is methylene diphenyl diisocyanate. In embodiments, the diisocyanate is toluene diisocyanate. In embodiments, the diisocyanate is hexamethylene diisocyanate. In embodiments, the diisocyanate is isophorone diisocyanate. In embodiments, the diisocyanate is tetramethylxylene diisocyanate.

In embodiments, the diisocyanate is: OCN—CH₂—NCO; OCN—(CH₂)₅—NCO; OCN—(CH₂)₇—NCO;

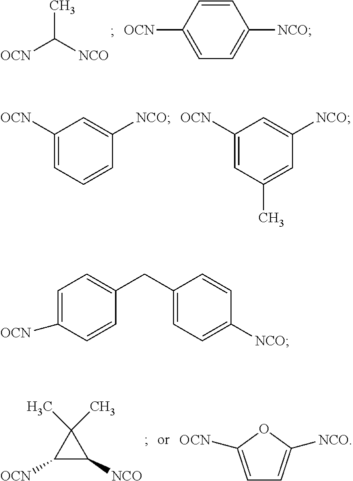

In embodiments, the acyl hydrazide has the formula:

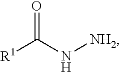

where R¹ is as defined herein.

In embodiments, the acyl azide has the formula:

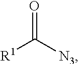

where R¹ is as defined herein.

In embodiments, the isocyanate is a monoisocyanate having the formula R¹—N═C═O, where R¹ is as defined herein.

R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is substituted or unsubstituted C₂-C₂₀alkyl, substituted or unsubstituted C₃-C₁₀cycloalkyl, substituted or unsubstituted C₅-C₆ aryl, or substituted or unsubstituted 5 or 6 membered heteroaryl. In embodiments, R¹ is substituted or unsubstituted C₂-C₂₀alkyl. In embodiments, R¹ is substituted or unsubstituted C₃-C₁₀cycloalkyl. In embodiments, R¹ is substituted or unsubstituted C₅-C₆ aryl. In embodiments, R¹ is substituted or unsubstituted 5 or 6 membered heteroaryl.

In embodiments, R¹ is R¹⁰⁰-substituted or unsubstituted alkyl (e.g., C₁-C₈, C₁-C₆, or C₁-C₄), R¹⁰⁰-substituted or unsubstituted heteroalkyl (e.g., 2 to 10 membered, 2 to 8 membered, 2 to 6 membered heteroalkyl, or 2 to 4 membered heteroalkyl), R¹⁰⁰-substituted or unsubstituted cycloalkyl (e.g., C₃-C₈, C₄-C₈, or C₅-C₆), R¹⁰⁰-substituted or unsubstituted heterocycloalkyl (e.g., 3 to 8 membered, 4 to 8 membered, or 5 to 6 membered), R¹⁰⁰-substituted or unsubstituted aryl (e.g., C₅-C₁₀ or C₅-C₆), or R¹⁰⁰-substituted or unsubstituted heteroaryl (e.g., 5 to 10 membered, 5 to 9 membered, or 5 to 6 membered). In embodiments, R¹ is R¹⁰⁰-substituted or unsubstituted alkyl (e.g., C₁-C₈, C₁-C₆, or C₁-C₄). In embodiments, R¹ is R¹⁰⁰-substituted or unsubstituted heteroalkyl (e.g., 2 to 10 membered, 2 to 8 membered, 2 to 6 membered heteroalkyl, or 2 to 4 membered heteroalkyl). In embodiments, R¹ is R¹⁰⁰-substituted or unsubstituted cycloalkyl (e.g., C₃-C₈, C₄-C₈, or C₅-C₆). In embodiments, R¹ is R¹⁰⁰-substituted or unsubstituted heterocycloalkyl (e.g., 3 to 8 membered, 4 to 8 membered, or 5 to 6 membered). In embodiments, R¹ is R¹⁰⁰-substituted or unsubstituted aryl (e.g., C₅-C₁₀ or C₅-C₆). In embodiments, R¹ is R¹⁰⁰-substituted or unsubstituted heteroaryl (e.g., 5 to 10 membered, 5 to 9 membered, or 5 to 6 membered).

R¹⁰⁰ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, R¹⁰¹-substituted or unsubstituted alkyl (e.g., C₁-C₈, C₁-C₆, or C₁-C₄), R¹⁰¹-substituted or unsubstituted heteroalkyl (e.g., 2 to 10 membered, 2 to 8 membered, 2 to 6 membered, or 2 to 4 membered), R¹⁰¹-substituted or unsubstituted cycloalkyl (e.g., C₃-C₈, C₄-C₈, or C₅-C₆), R¹⁰¹-substituted or unsubstituted heterocycloalkyl (e.g., 3 to 8 membered, 4 to 8 membered, or 5 to 6 membered), R¹⁰¹-substituted or unsubstituted aryl (e.g., C₅-C₁₀ or C₅-C₆), or R¹⁰¹-substituted or unsubstituted heteroaryl (e.g., 5 to 10 membered, 5 to 9 membered, or 5 to 6 membered).

R¹⁰¹ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl (e.g., C₁-C₈, C₁-C₆, or C₁-C₄), unsubstituted heteroalkyl (e.g., 2 to 10 membered, 2 to 8 membered, 4 to 8 membered, 2 to 6 membered, or 2 to 4 membered), unsubstituted cycloalkyl (e.g., C₃-C₈, C₄-C₈, or C₅-C₆), unsubstituted heterocycloalkyl (e.g., 3 to 8 membered, 4 to 8 membered, or 5 to 6 membered), unsubstituted aryl (e.g., C₅-C₁₀ or C₅-C₆), or unsubstituted heteroaryl (e.g., 5 to 10 membered, 5 to 9 membered, or 5 to 6 membered).

In embodiments, R¹ is unsubstituted C₁-C₂₀alkyl. In embodiments, R¹ is unsubstituted C₁-C₁₆alkyl. In embodiments, R¹ is unsubstituted C₁-C₁₂alkyl. In embodiments, R¹ is unsubstituted C₁-C₁₀alkyl. In embodiments, R¹ is unsubstituted C₁-C₈alkyl. In embodiments, R¹ is unsubstituted 5 or 6 membered heteroaryl. In embodiments, R¹ is a C₅-C₆ aryl or 5 or 6 membered heteroaryl fused to a C₅-C₆ aryl or 5 or 6 membered heteroaryl. In embodiments, R¹ is a C₅-C₆ aryl or 5 or 6 membered heteroaryl fused to C₅-C₆ aryl or 5 or 6 membered heteroaryl fused to a C₅-C₆ aryl or 5 or 6 membered heteroaryl.

In embodiments, R¹ is —(CH₂)₅CH₃. In embodiments, R¹ is —(CH₂)₁₄CH₃. In embodiments, R¹ is —(CH(CH₂CH₃))(CH₂)₃CH₃. In embodiments, R¹ is

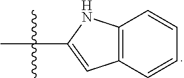 [SMILES: *c1cc2ccccc2[nH]1]

In embodiments, R¹ is

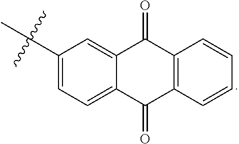 [SMILES: *c1ccc2c(c1)C(=O)c1ccccc1C2=O]

In embodiments, R¹ is

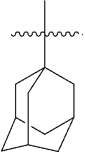 [SMILES: *C12CC3CC(CC(C3)C1)C2]

In embodiments, the monoisocyanate is CH₃(CH₂)₅NCO; CH₃(CH₂)₁₄NCO;

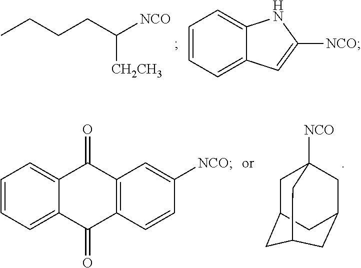

The isocyanates, such as the diisocyanates, described herein can be used to make polyurethanes. In embodiments, the diisocyanate described herein are reacted with a polyol to produce polyurethane. Methods for producing polyurethane from diisocyanates are known in the art.

The disclosure provides methods of producing polyurethane by a process comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) heating the third solution comprising the azide compound in flow to produce a diisocyanate; and (iv) reacting the diisocyanate with a polyol to produce the polyurethane. In embodiments, the method comprises reacting the diisocyanate with a polyol in the presence of a catalyst to produce the polyurethane. In embodiments, the method comprises reacting the diisocyanate with a polyol by activation with ultraviolet light to produce the polyurethane. Any polyol known in the art of polyurethane production can be used in the methods described herein. In embodiments, the polyol is hydroxyl-terminated polyether. or a hydroxyl-terminated polyester. Any catalyst known in the art of polyurethane production can be used in the methods described herein. In embodiments the catalyst is a tin carboxylate, an amine, a bismuth carboxylate, a zinc carboxylate, a zirconium carboxylate, or a nickel carboxylate.

The disclosure provides methods of producing polyurethane by a process comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) removing water from the second solution in flow to produce a third solution comprising the azide compound and the organic solvent; (iv) heating the third solution comprising the azide compound in flow to produce a diisocyanate; and (v) reacting the diisocyanate with a polyol to produce the polyurethane. In embodiments, the method further comprises drying the third solution in flow prior to heating the third solution, where drying the third solution removes residual water from the organic solvent prior to heating the third solution to perform the Curtius rearrangement. In embodiments, the method comprises reacting the diisocyanate with a polyol in the presence of a catalyst to produce the polyurethane. In embodiments, the method comprises reacting the diisocyanate with a polyol by activation with ultraviolet light to produce the polyurethane. Any polyol known in the art of polyurethane production can be used in the methods described herein. In embodiments, the polyol is hydroxyl-terminated polyether. or a hydroxyl-terminated polyester. Any catalyst known in the art of polyurethane production can be used in the methods described herein. In embodiments the catalyst is a tin carboxylate, an amine, a bismuth carboxylate, a zinc carboxylate, a zirconium carboxylate, or a nickel carboxylate.

The disclosure provides methods of producing polyurethane by a process comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) removing water from the second solution in flow to produce a third solution comprising the azide compound and the organic solvent; (iv) drying the third solution in flow; (v) heating the third solution comprising the azide compound in flow to produce the isocyanate; and (vi) reacting the diisocyanate with a polyol to produce the polyurethane. In embodiments, the method comprises reacting the diisocyanate with a polyol in the presence of a catalyst to produce the polyurethane. In embodiments, the method comprises reacting the diisocyanate with a polyol by activation with ultraviolet light to produce the polyurethane. Any polyol known in the art of polyurethane production can be used in the methods described herein. In embodiments, the polyol is hydroxyl-terminated polyether. or a hydroxyl-terminated polyester. Any catalyst known in the art of polyurethane production can be used in the methods described herein. In embodiments the catalyst is a tin carboxylate, an amine, a bismuth carboxylate, a zinc carboxylate, a zirconium carboxylate, or a nickel carboxylate.

### Embodiments 1 to 56

Embodiment 1. A continuous flow process for producing an isocyanate, the process comprising: (i) mixing an acyl hydrazide with an aqueous solution comprising nitrous acid in flow to form a first solution comprising an acyl azide and water; (ii) mixing an organic solvent with the first solution in flow to produce a second solution comprising the acyl azide compound, the organic solvent, and water; (iii) removing water from the second solution in flow to produce a third solution comprising the azide compound and the organic solvent; and (iv) heating the third solution comprising the azide compound in flow to produce the isocyanate.

Embodiment 2. The process of Embodiment 1, further comprising drying the third solution in flow prior to heating the third solution.

Embodiment 3. A process for producing at least one gram of an isocyanate, the process comprising: (i) mixing an acyl hydrazide with an aqueous solution comprising nitrous acid to form a first solution comprising an acyl azide and water; (ii) mixing an organic solvent with the first solution to produce a second solution comprising the azide compound, the organic solvent, and water; (iii) removing water from the second solution to produce a third solution comprising the azide compound and the organic solvent; and (iv) heating the third solution comprising the azide compound to produce at least one gram of the isocyanate.

Embodiment 4. The process of Embodiment 1, further comprising drying the third solution prior to heating the third solution.

Embodiment 5. The process of any one of Embodiments 1 to 4 for producing at least one kilogram of the isocyanate.

Embodiment 6. The process of Embodiment 5 for producing at least 500 kilograms of an isocyanate.

Embodiment 7. The process of any one of Embodiments 1 to 6, wherein the organic solvent comprises chloroform, toluene, benzene, methyl tert-butyl ether, tetrahydrofuran, or a mixture of two or more thereof.

Embodiment 8. The process of any one of Embodiments 1 to 7, wherein the ratio of acyl hydrazide to nitrous acid is from about 1:1 to about 1:3.

Embodiment 9. The process of Embodiment 8, wherein the ratio of acyl hydrazide to nitrous acid is from about 1:2.

Embodiment 10. The process of any one of Embodiments 1 to 9, wherein the volume of organic solvent to the volume of the first solution is from about 2:1 to about 1:2.

Embodiment 11. The process of Embodiment 10, wherein the volume of organic solvent to the volume of the first solution is from about 1:1.

Embodiment 12. The process of any one of Embodiments 1 to 11, comprising mixing the acyl hydrazide with the aqueous solution in flow at a temperature from about 0° C. to about 30° C.

Embodiment 13. The process of Embodiment 12, comprising mixing the acyl hydrazide with the aqueous solution in flow at a temperature from about 20° C. to about 25° C.

Embodiment 14. The process of any one of Embodiments 1 to 13, comprising heating the third solution in flow at a temperature from about 65° C. to about 120° C.

Embodiment 15. The process of Embodiment 14, comprising heating the third solution in flow at a temperature from about 85° C. to about 100° C.

Embodiment 16. The process of any one of Embodiments 1 to 15, wherein the isocyanate is a diisocyanate.

Embodiment 17. The process of Embodiment 16, wherein the acyl hydrazide has the structure:

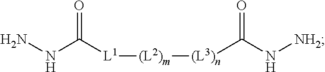

wherein the acyl azide has the structure:

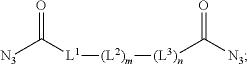

and wherein the isocyanate has the structure:
O═C═N-L¹-(L²)ₘ-(L³)ₙ-N═C═O; wherein L¹, L², and L³ are each independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; m is 0 or 1; and n is 0 or 1.

Embodiment 18. The process of Embodiment 17, wherein L¹, L², and L³ are each independently substituted or unsubstituted C₁-C₁₂alkylene, substituted or unsubstituted C₅-C₆ cycloalkylene, substituted or unsubstituted C₅-C₆ arylene, or substituted or unsubstituted 5 or 6 membered heteroarylene.

Embodiment 19. The process of Embodiment 17 or 18, wherein m and n are 0.

Embodiment 20. The process of Embodiment 17 or 18, wherein m and n are 1.

Embodiment 21. The process of Embodiment 17 or 18, wherein m is 1 and n is 0.

Embodiment 22. The process of Embodiment 16, wherein the diisocyanate is methylene diphenyl diisocyanate.

Embodiment 23. The process of Embodiment 16, wherein the diisocyanate is toluene diisocyanate.

Embodiment 24. The process of Embodiment 16, wherein the diisocyanate is hexamethylene diisocyanate.

Embodiment 25. The process of Embodiment 16, wherein the diisocyanate is isophorone diisocyanate.

Embodiment 26. The process of Embodiment 16, wherein the diisocyanate is tetramethylxylene diisocyanate.

Embodiment 27. The process of Embodiment 16, wherein the diisocyanate is:

OCN—CH₂—NCO; OCN—(CH₂)₅—NCO; OCN—(CH₂)₇—NCO;

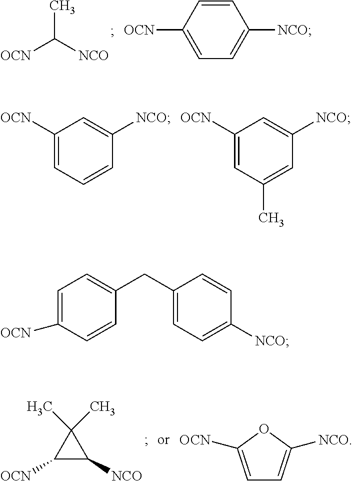

Embodiment 28. The process of any one of Embodiments 1 to 15, wherein the isocyanate is a monoisocyanate.

Embodiment 29. The process of Embodiment 28, wherein the acyl hydrazide has the structure:

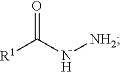

wherein the acyl azide has the structure:

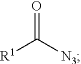

and wherein the isocyanate has the structure: R¹—N═C═O; wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 30. The process of Embodiment 29, wherein R¹ is substituted or unsubstituted C₂-C₂₀alkyl, substituted or unsubstituted cycloalkyl, substituted or unsubstituted C₅-C₆ aryl, or substituted or unsubstituted 5 or 6 membered heteroaryl.

Embodiment 31. The process of Embodiment 28, wherein the monoisocyanate is:

CH₃(CH₂)₅NCO; CH₃(CH₂)₁₄NCO;

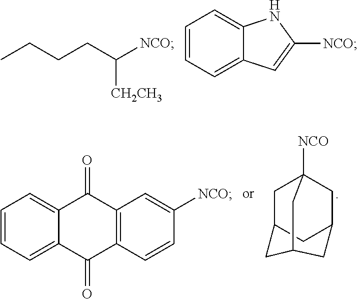

### Embodiments P1 to P12

Embodiment P1. A process for producing diisocyanate, the process comprising: (i) converting a carboxylic acid to an ester through Fischer esterification; (ii) converting the ester to a hydrazide with hydrazine hydrate; and (iii) utilizing flow chemistry to convert the hydrazide to a diisocyanate through Curtius rearrangement.

Embodiment P2. The process of Embodiment P1, wherein the carboxylic acid is azelaic acid, heptanoic acid, pimelic acid, terephthalic acid, or a combination thereof.

Embodiment P3. The process of Embodiment P2, wherein azelaic acid and heptanoic acid are synthesized from algae oil.

Embodiment P4. The process of Embodiment P3, wherein the algae oil is obtained from Nannochloropsis salina.

Embodiment P5. The process of any one of Embodiments P1 to P4, wherein the diisocyanate is OCN—(CH₂)₇—NCO, CH₃(CH₂)₅—NCO, OCN—(CH₂)₅—NCO, or

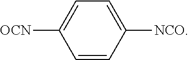

Embodiment P6. A process for producing heptamethylene diisocyanate, the process comprising: (i) converting azaleic acid to azelaic acid ester through Fischer esterification; (ii) converting the azelaic acid ester to azelaic acid dihydrazide with hydrazine hydrate; and (iii) utilizing flow chemistry and Curtius rearrangement to convert azelaic acid dihydrazide to a nonanedioyl diazide by nitric acid, and to convert the nonanedioyl diazide to heptamethylene diisocyanate.

Embodiment P7. The process of Embodiment P6, wherein the azalaic acid is obtained from algae-sourced palmitoleic acid through ozonolysis.

Embodiment P8. The process of Embodiment P7, wherein the algae-sourced palmitoleic acid is obtained from Nannochloropsis salina.

Embodiment P9. The process of any one of Embodiments P6 to P8, wherein the flow chemistry utilizes toluene to extract the nonanedioyl diazide

Embodiment P10. The process of any one of Embodiments P1 to P9, wherein the Curtius rearrangement is conducted at a temperature from about 75° C. to about 95° C.

Embodiment P11. The process of Embodiment P10, wherein the Curtius rearrangement is conducted at a temperature of about 85° C.

Embodiment P12. The process of any one of Embodiments P1 to P11, wherein the process is a batch process.

### EXAMPLES

The following examples are for purposes of illustration and is not intended to limit the spirit or scope of the disclosure or claims.

Continuous flow processes can provide improved reaction safety, accelerated reaction kinetics, clean production methods, and improved scale-up potential. (Refs 10-12). While other studies have explored the Curtius rearrangement in continuous flow, none has mitigated the safety challenges of acyl azide preparation. (Refs. 9-11, 13). Here, the inventors developed a new strategy to prepare acyl azides in flow coupled with Curtius rearrangement. The inventors applied this solution first to the preparation of aliphatic diisocyanates derived from algae biomass, a renewable resource with the lowest environmental impact among petroleum replacements. The process was then expanded for application to linear alkyl and aromatic isocyanates from available organic acids and diacids and demonstrated facile scaleup capability. This methodology is safe, scalable, and is amenable to distributed production using renewable feedstocks.

### Example 1

Preparation of Mono- and Diisocyanates in Flow from Renewable Carboxylic Acids

This study began with our desire to prepare 1,7-heptamethylene diisocyanate (HpMDI) from azelaic acid (AA), a 9-carbon diacid that we derived from algae-sourced palmitoleic acid via ozonolysis. (Ref 14). We saw an opportunity using the Curtius rearrangement to achieve this transformation, but preparation of the requisite diacyl azide intermediate presented stability concerns. (Refs. 13, 15, 16). For this reason, the inventors hypothesized that continuous flow chemistry offered an ideal methodology, where the risks of unstable reactions or intermediates can be mitigated. (Refs. 9-11, 13, 17, 18). We reasoned that we could prepare azelaoyl azide from azidination of the more stable nonanedihydrazide with nitrous acid in flow and couple this to an in-flow Curtius rearrangement.

The recent publication of several new tools for laboratory-scale flow chemistry, often through open-source, has significantly lowered the barrier to entry for these methods by building a simple, low-priced flow chemistry system based on readily available units such as stepper-motors, Arduino controls, and 3D printed parts. (Ref 19). This system can also be customized and incorporated with liquid-liquid separators (Zaiput, Waltham, Mass.) to satisfy research needs specific to the laboratory setting.

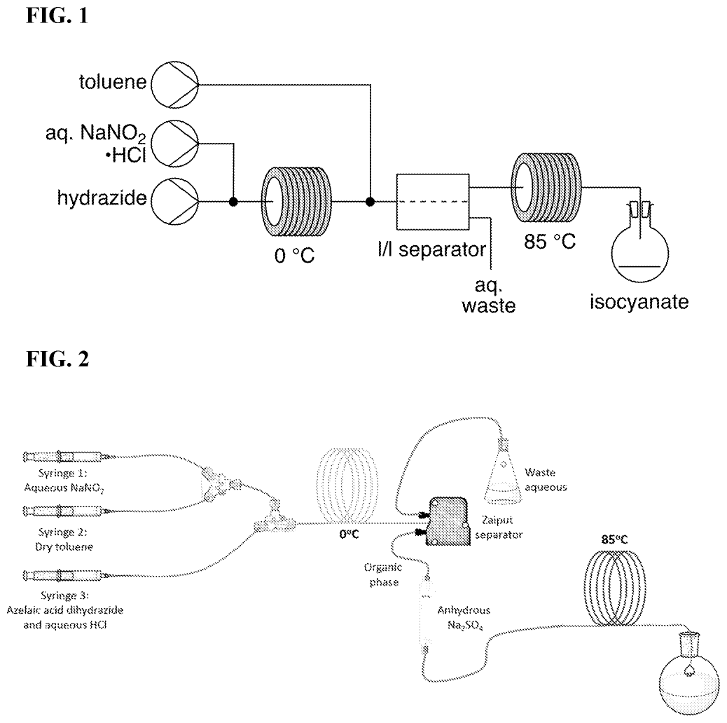

To achieve the transformation of azelaic acid (AA) to 1,7-heptamethylene diisocyanate, AA was first converted to azelaic dimethyl ester by Fischer esterification, followed by in situ treatment with hydrazine hydrate, whereupon the nonanedihydrazide is collected by filtration. (Refs. 20, 21). The first step in scheme 1 to collect nonanedihydrazide from azelaic acid was performed in batch chemistry. Intermediate nonanedihydrazide was converted to the azelaoyl azide in continuous flow (FIG. 1) at 0° C. with addition of nitrous acid. Toluene was then added to the flow, and the organic-soluble azelaoyl azide was extracted into the organic phase using a Zaiput separator. (Ref 22). Further drying of azelaoyl azide in toluene is accomplished with an in-line sodium sulfate column, followed by heating in flow at 85° C. to affect the Curtius rearrangement and produce 1,7-heptamethylene diisocyanate in 80% yield at a rate of 500 mg h⁻¹.

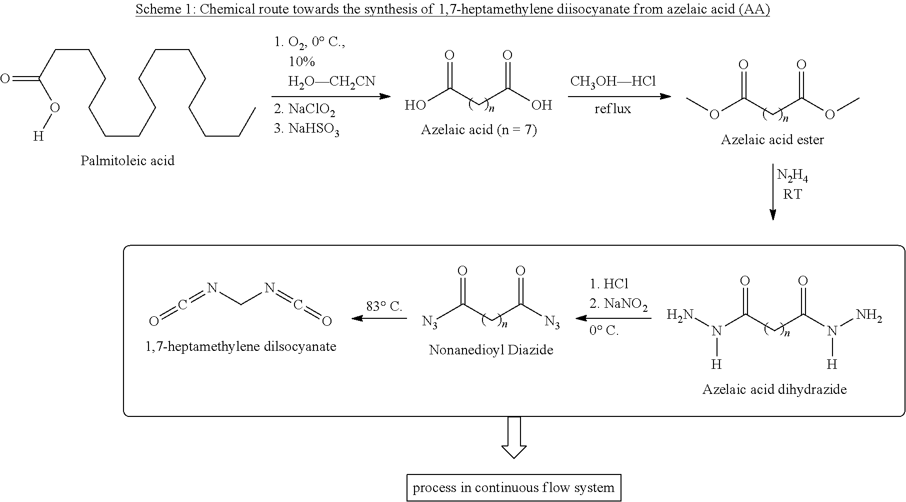

This route addresses several bottlenecks at once. First, we chose to prepare the acyl hydrazide in situ following Fischer esterification. (Refs. 20, 21, 23-26). While a flow process could be utilized for this step, it has been reported that product precipitation out of methanol drives the reaction to completion. (Refs. 20, 21, 27-32). This could result in clogging of a flow mixer, channel, or pressure regulator. (Ref 33). We found batch methods to be convenient and reproducible, and experimental conditions for both esterification and acyl hydrazide synthesis were carried out based on previous reports. (Refs. 20, 21, 24-26, 32, 34). While hydrazine is a toxic and unstable compound, hydrazine hydrate can be safely handled and diluted in methanol to prepare hydrazide derivatives in batch. (Ref 35). Transformation of acyl hydrazide to isocyanate presents the most critical safety concern due to the instability of the high-energy acyl azide intermediate. (Refs. 15, 16). Here flow chemistry is a preferable choice, since a flow reactor can prepare very small quantities of hazardous intermediates, with efficient stabilizing conditions such as heat exchange, pressure regulation, and residence time, thereby avoiding the accumulation of hazardous, explosive materials. (Refs. 9, 11, 13, 33, 36).

In addition, the acyl hydrazide is soluble in water, enabling safe oxidation with nitrous acid in aqueous flow conditions. The resulting acyl azide is then immediately extracted into the organic phase in flow. Finally, acyl azide conversion into diisocyanate can be carried out at high temperature in safe and controlled flow conditions, without intermediate isolation. Taken together, this process can be carried out in any laboratory and may be scaled with limited risk. In demonstrating this continuous flow methodology, we found that it offered safe material handling that facilitated in situ formation of Curtius precursors and rearrangement of resulting hazardous intermediates in flow, for an improved and potentially scalable route to isocyanates. (Refs. 37, 38).

Given the success with small-scale flow apparatus, we decided to scale this process on an instrument with a higher flow rate. (Refs. 39, 40). Using a Vapourtec R-series flow apparatus at 1-2 mL min⁻¹, we could perform continuous production of 1,7-heptamethylene diisocyanate at 10 g h⁻¹ scale. The small Zaiput separator was not compatible with the higher production rate, and a work-around for the liquid-liquid separator was built using common lab glassware. This consisted of flowing into an uneven U-shaped glass tube open to the atmosphere where the phases separate. The lower density toluene layer containing the acyl azide reaches a higher level on one side and runs out of the U-shaped tube into a catchment flask. It is then withdrawn and pumped through a drying column for subsequent Curtius rearrangement in flow. The aqueous layer overflows at the entry side of the U-shaped tube and is discarded. Compared to a phase separation unit, this separation system does not require a pump or impedance apparatus to keep the balance between the levels of two-phases. (Ref 13). We found it to offer reliable setup that permitted high flow rate with excellent separation. Here, fluid levels remained constant regardless of flow rate, allowing the preparation of 1,7-heptamethylene diisocyanate at 8.5 g h⁻¹ and 90% yield. We used this setup to prepare 100 g of heptamethylene diisocyanate from algae-sourced azelaic acid, a 9-carbon diacid derived from palmitoleic acid via ozonolysis. (Ref 14).

Many factors affecting the yield and purity of Curtius rearrangement products have been reported, including as reaction kinetics (Refs. 41-43), solvents (Refs. 9, 11, 38, 44, 45), temperature (Refs 11, 38, 46-48), calorimetry (Refs. 49, 50), energy release (Ref 13), residence time (Refs 13, 37), and analytics (Ref 13). We found that the use of toluene provided optimal rearrangement conditions at 85° C. (Refs. 11, 38, 46). Due to the potentially explosive and hazardous nature of in situ acyl azide intermediates, appropriate concentration of acyl hydrazide, reactor volume and flow, and residence time should be carefully considered to control energy release. Heat release can be attributed to the decomposition of the acyl azide to form the isocyanate and nitrogen, which is dependent on the concentration of the intermediate and temperature. (Ref 49). Differential Scanning calorimetry (DSC) indicated that 0.5M of acyl azide releases a heat energy of 187 J/g, while 1M of acyl azide liberates an energy of 364 J/g which exceeds safety criteria. (Refs. 13, 50, 51). Based on calorimetric data studies from previous reports (Refs. 13, 49-51), we chose hydrazide concentrations held at 0.1M, with a reactor volume for the Curtius rearrangement of 1.6 mL and residence time of 97 seconds, keeping energy release at a minimum for improved safety. In a flow system, the thermal stability of acyl azides generated in situ are minimized due to solvent dilution during the flow reaction. To further minimize risks when moving to larger scale, we controlled the organic phase volume during separation by selecting a U-shape tube of appropriate size to never exceed 20 mL, representing a maximum of 1 g of intermediate azelaoyl azide throughout the flow process.

The general methodology of using U-shape tube may be applied to the preparation of any alkyl or aromatic isocyanate (Scheme 2). The generality of this process was evaluated with a series of mono- and dicarboxylic acids (Table 1). In a typical experiment, the carboxylic acid is first converted to the hydrazide derivative in batch, as described. Using a small-scale flow apparatus, we converted 16 hydrazides to their corresponding isocyanate derivatives using a tandem acyl azide formation—Curtius rearrangement in flow, without isolating the high-energy acyl azides. From these data, we found that the secondary and tertiary aliphatic and aromatic carboxylic acids provide slightly reduced yields, however our findings suggest that optimization is possible through the modification of reaction conditions. Based on the data reported in Table 1, primary aliphatic hydrazides provide the highest yields. Secondary aliphatic hydrazides provide yields similar to aromatics, followed by tertiary aliphatics. This indicates that sterics plays a major role in conversion to isocyanates, in keeping with the current mechanistic understanding of the thermal Curtius rearrangement, which is reported to proceed through a concerted alkyl migration of the acyl nitrene following loss of nitrogen from the acyl azide. (Refs. 16, 31, 52-54).

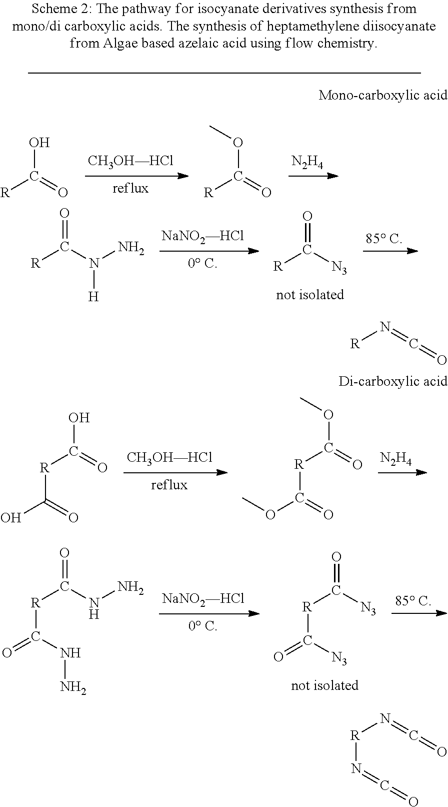

TABLE 1
Synthesis of isocyanates from carboxylic acids

| Entry | Substrate | Product | Yield % |
|---|---|---|---|
| 1 | HOOC—CH₂—COOH | OCN—CH₂—NCO | 80 |
| 2 | HOOC—(CH₂)₅—COOH | OCN—(CH₂)₅—NCO | 82 |
| 3 | HOOC—(CH₂)₇—COOH | OCN—(CH₂)₇—NCO | 90 |
| 4 | CH₃(CH₂)₅COOH | CH₃(CH₂)₅NCO | 70 |
| 5 | CH₃(CH₂)₁₄COOH | CH₃(CH₂)₁₄NCO | 66 |
| 6 | 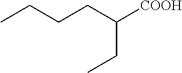 | 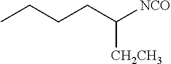 | 55 |
| 7 | 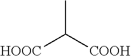 | 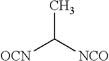 | 50 |
| 8 | 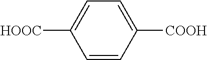 [SMILES: O=C(O)c1ccc(C(=O)O)cc1] | 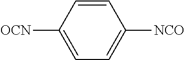 | 68 |
| 9 | 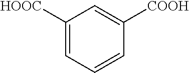 [SMILES: O=C(O)c1cccc(C(=O)O)c1] | 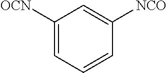 | 72 |
| 10 | 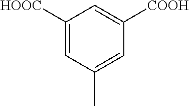 | 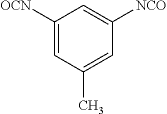 | 70 |
| 11 | 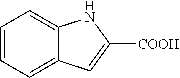 [SMILES: O=C(O)c1cc2ccccc2[nH]1] | 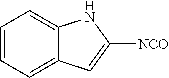 | 61 |
| 12 | 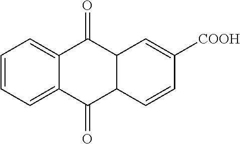 [SMILES: O=C(O)C1=CC2C(=O)c3ccccc3C(=O)C2C=C1] | 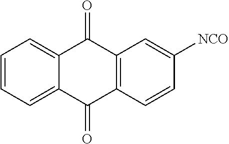 | 65 |
| 13 | 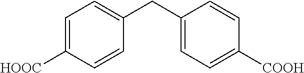 [SMILES: O=C(O)c1ccc(Cc2ccc(C(=O)O)cc2)cc1] | 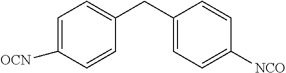 | 72 |
| 14 | 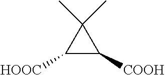 | 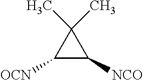 | 75 |
| 15 | 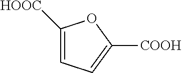 [SMILES: O=C(O)c1ccc(C(=O)O)o1] | 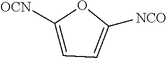 | 73 |
| 16 | 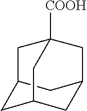 [SMILES: O=C(O)C12CC3CC(CC(C3)C1)C2] | 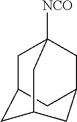 | 51 |

The present methodology holds particular promise for the production of isocyanates from natural sources, in which carboxylic acids predominate as available functionalities for conversion. As with azelaic acid, several derivatives in Table 1 can be generated from natural sources, including entries 1-5, 14, and 15, and we have explored the development of algae-based sources for application to polyurethanes in our laboratory. While isocyanates have been prepared from renewable sources using contemporary methods, algae-based isocyanates have not been explored until now. (Refs. 47, 48, 55). In addition, the present methodology is amenable to production of aromatic isocyanates, which have significant applications in commercial products. As examples, 2,5-furandicarboxylic acid (entry 15, Table 1) is derived from renewable monosaccharides, and (1R,2R′)-3,3-dimethyl-1,2-cyclopropanedicarboxylic acid (entry 14, Table 1) derives from ozonolysis of chrysanthemic acid. This route now provides the opportunity for production of sustainable polyurethanes prepared from polyols and isocyanates that can both be derived from algae oil. In addition, the present method can be applied to the practice of distributed manufacturing, such that isocyanates can be safely prepared at a variety of scales and locations without the need for capital-intensive phosgene generation and containment facilities. (Ref 56)

In summary, we have designed a procedure for the synthesis of both aliphatic and aromatic diisocyanates from carboxylic acids using simple flow chemistry methodologies. This transformation is safe, scalable, and distributable, and should have ability to transform sustainable carboxylic acids to their corresponding isocyanates. As an application, we demonstrated the scaled production (100 g) of 1,7-heptamethylene diisocyanate utilizing a dicarboxylic acid derived from algae oil, which is among the most sustainable bioresources available. Finally, the use of continuous flow chemistry, a safe and scalable approach, provides a route to diisocyanates through Curtius rearrangement that in principal can be modified for use in manufacture on an industrial scale.

Materials and Methods

Chemical reagents were purchased from Fisher Chemical, Macron Fine Chemicals, Sigma Aldrich, Acros, Fluka, Alfa Aesar Chemicals or TCI. All chemicals were regent grade and used without further purification. Analytical grade solvents such as acetone, hexane, acetonitrile, dichloromethane, ethanol and methanol were purchased from Fisher Chemical and used as received. Deuterated NMR solvents such as chloroform-d, DMSO-d6 were purchased from Cambridge Isotope Laboratories.

Gas chromatography mass spectrometry (GC-MS) was measured using an Agilent 7890A GC system connected to a 5975C VL MSD quadrupole MS (EI). Samples were separated on a 60 m DB23 Agilent GCMS column using helium as carrier gas and a gradient of 110° C. to 200° C. at 15° C. min⁻¹, followed by 20 minutes at 200° C. ¹H NMR and ¹³C NMR spectra were recorded on a JOEL ECA 500 or a Varian VX500 spectrometer equipped with an Xsens Cold probe. ThermoFinnigan LCQ Deca spectrometer was used for Electrospray (ESI) mass spectrometric analyses while ThermoFinnigan MAT900XL mass spectrometer with electron impact (EI) ionization was used for high resolution analyses. High resolution electrospray ionization mass spectrometry analyses (HR-ESI-MS) were performed using a Thermo Scientific LTQ Orbitrap XL mass spectrometer. FT-IR was recorded on PerkinElmer FTIR. Ozonolysis is conducted in batch mode with the Triogen LAB2B Ozone generator. Flow chemistry system is constructed and assembled from four different parts: building syringe pumps, connecting the electronics, designing and connecting the tubing and Arduino code options. This system is set up followed the instruction and reference from Croatt Research group (University of North Carolina at Greensboro). To perform the studies described herein we constructed a flow system analogous to those from the Croatt Research group. For this set up system, it is possible to modify variable power supplies for stepper motors with a higher torque rating, a necessity for varying syringe volumes. Moreover, the instruction for installing Zaiput separator and drying unit can be referenced from followed paper (Britton, J.; Jamison, T. F. The assembly and use of continuous flow systems for chemical synthesis. Nat. Protoc. 2017, 12, 2423-2447). Continuous flow experiments were carried out using a Vapourtec R Series Flow Chemistry system (Dhanya, R. P.; Herath, A.; Sheffler, D. J.; Cosford, N. D. P. A combination of flow and batch mode processes for the efficient preparation of mGlu2/3 receptor negative allosteric modulators (NAMs). Tetrahedron 2018, 74, 3165-3170; Herath, A.; Cosford, N. D. P. Continuous-flow synthesis of highly functionalized imidazo-oxadiazoles facilitated by microfluidic extraction. Beilstein J. Org. Chem. 2017, 13, 239-246) for scaling up products.

Synthesis of Azelaic acid and Heptanoic acid from algae oil: Preparing according to methods previously reported by our lab (Hai, T. A. P.; Neelakantan, N.; Tessman, M.; Sherman, S. D.; Griffin, G.; Pomeroy, R.; Mayfield, S. P.; Burkart, M. D. Flexible polyurethanes, renewable fuels, and flavorings from a microalgae oil waste stream. Green Chem. 2020, 10. 1039/d0 gc00852d) including purification of fatty acids from biomass, pigment removal, isolation of mono-unsaturated fatty acid C16-1, and oxidative cleavage of C16-1 to synthesize azelaic acid and heptanoic acid.

Azelaic acid: ¹H NMR (500 MHz, DMSO-d₆) δ=11.95 (s, 1H), 2.15 (t, J=7.3, 2H), 1.44 (p, J=7.1, 2H), 1.21 (d, J=6.5, 3H); ¹³C NMR (126 MHz, DMSO-d₆) δ=175.10, 34.16, 28.95, 24.97; HR-ESI-MS calculated for C₉H₁₆O₄[M−H]⁻: 187.22, found 187.25

Heptanoic acid: ¹H NMR (500 MHz, DMSO-d₆) δ=11.92 (s, 1H), 2.14 (t, J=7.3, 2H), 1.44 (q, J=7.1, 2H), 1.27-1.18 (m, 7H), 0.85-0.79 (m, 2H); ¹³C NMR (126 MHz, DMSO-d₆) δ=175.17, 34.18, 31.48, 29.53, 28.72, 24.97, 22.49, 14.38; HR-ESI-MS calculated for C₇H1402 [M−H]⁻: 129.18, found 129.23

Synthesis of Azelaic acid ester: Azelaic acid (20 g, 0.1 mol) was added to a 250 mL one-neck round-bottom flask, then 86 mL methanolic HCl 1M was poured into this flask (Al-Amiery, A. A.; Kassim, F. A. B.; Kadhum, A. A. H.; Mohamad, A. B. Synthesis and characterization of a novel eco-friendly corrosion inhibition for mild steel in 1M hydrochloric acid. Scientific Reports 2016, 6, 19890). The reaction was reflux for 2 h at temperature of 80° C. Once finishing reaction, 100 mL hexane was added to the flask. Two layers were divided by separating funnel. The hexane layer on top was collected, dried over anhydrous Na₂SO₄, and removed solvent to obtain azelaic acid ester as a colorless oil with the yield of 82%.

Dimethyl azelate: ¹H NMR (500 MHz, Chloroform-d) δ=3.64 (s, 3H), 2.28 (t, 2H), 1.59 (t, J=7.2, 2H), 1.29 (s, 2H), 1.33-1.22 (m, 1H).

Synthesis of Azelaic acid dihydrazide: 14.5 mL N₂H₄ (0.5 mol) was added slowly to 80 mL methanol containing 10 g azelaic acid ester (0.05 mol) in 150 mL one-neck round-bottom flask (Al-Amiery, A. A.; Kassim, F. A. B.; Kadhum, A. A. H.; Mohamad, A. B. Synthesis and characterization of a novel eco-friendly corrosion inhibition for mild steel in 1M hydrochloric acid. Scientific Reports 2016, 6, 19890; Poolman, J. M.; Maity, C.; Boekhoven, J.; Mee, L. v. d.; Sage, V. A. A. 1; Groenewold, G. J. M.; Kasteren, S. I. v.; Versluis, F.; Esch, J. H. v.; Eelkema, R. A toolbox for controlling the properties and functionalisation of hydrazone-based supramolecular hydrogels. J. Mater. Chem. B 2016, 4, 852-859; Cougnon, F. B. L.; Caprice, K.; Pupier, M.; Bauzá, A.; Frontera, A. A Strategy to Synthesize Molecular Knots and Links Using the Hydrophobic Effect. J. Am. Chem. Soc. 2018, 140, 12442-12450). A white slurry was observed after refluxing the mixture for 6 h at a temperature of 90° C. Azelaic acid dihydrazide (88% in yield) appeared as a white powder was collected after filtration and washing with methanol.

Azelaic acid dihydrazide: ¹H NMR (500 MHz, DMSO-d₆) δ=8.88 (s, 1H), 4.13 (s, 2H), 1.95 (t, J=7.4, 2H), 1.42 (p, J=7.2, 6.7, 2H), 1.18 (s, 3H), 1.15 (d, J=7.5, 1H); ¹³C NMR (126 MHz, DMSO-d₆) δ=25.73, 31.24, 33.93, 172.15; HR-ESI-MS calculated for C₉H₂₀N₄O₂[M+H]⁺: 217.28, found 217.16

Flow chemistry for synthesis of heptamethylene diisocyanate: The flow system was set up as described in FIG. 2.

Solution 1: sodium nitrite (1.37 g, 0.02 mol) was distilled into 100 mL deionized water. The concentration was 0.2M

Solution 2: pure toluene

Solution 3: azelaic dihydrazide (2.16 g, 0.01 mol) was dissolved in 100 mL deionized water and 0.8 mL conc. HCl. The concentration was 0.1M.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing heptamethylene diisocyanate product was placed in an Erlenmeyer flask. After removing solvent, heptamethylene diisocyanate was collected as a pale-yellow liquid and stored at a temperature of 4° C. This system provides heptamethylene diisocyanate at a rate of 500 mg h⁻¹.

Heptamethylene diisocyanate: ¹H NMR (500 MHz, Chloroform-d) 6=3.29 (t, J=6.6, 5H), 2.21 (t, J=7.5, 0H), 1.60 (h, J=6.1, 6H), 1.44-1.27 (m, 7H); ¹³C NMR (126 MHz, Chloroform-d) δ=138.02, 129.21, 128.40, 125.47, 122.13, 43.05, 31.33, 31.29, 28.49, 26.55, 21.60; HR-ESI-MS calculated for C₉H₁₄N₂O₂[M+H]⁺: 183.23, found 183.07

Synthesis of hexyl isocyanate from heptanoic acid originating from algae oil (Table 1, entry 4)

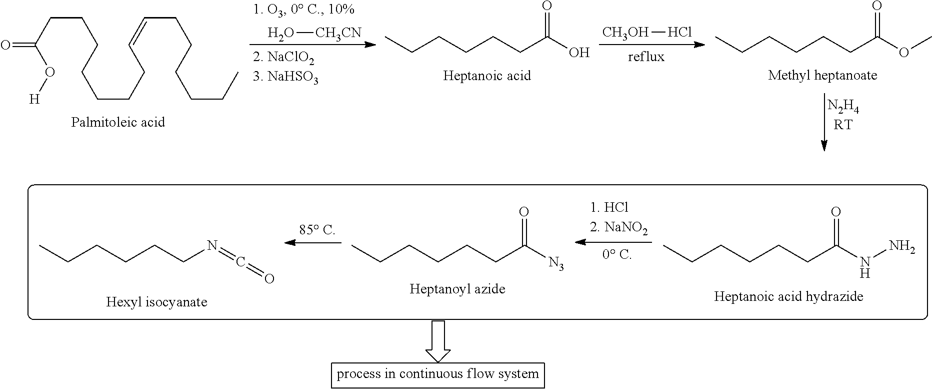

Synthesis of methyl heptanoate: Heptanoic acid (20 g, 0.15 mol) was added to a 250 mL one-neck round-bottom flask, then 125 mL methanolic HCl 1M was poured into this flask. The reaction was reflux for 2 h. Once finishing reaction, 100 mL hexane was added to the flask. After separation by funnel, the organic layer was dried over anhydrous Na₂SO₄, and evaporated to afford azelaic acid ester as a colorless liquid with the yield of 85%.

Methyl heptanoate: ¹H NMR (500 MHz, Chloroform-d) δ=3.64 (s, 3H), 2.31-2.25 (m, 2H), 1.59 (t, J=7.3, 2H), 1.34-1.21 (m, 12H), 0.90-0.81 (m, 4H).

Synthesis of heptanoic acid hydrazide: The methyl heptanoate (10 g, 0.06 mol) was dispersed in a mixture of methanol (140 mL) and hydrazine monohydrate (21 mL, 0.6 mol) (Al-Amiery, A. A.; Kassim, F. A. B.; Kadhum, A. A. H.; Mohamad, A. B. Synthesis and characterization of a novel eco-friendly corrosion inhibition for mild steel in 1M hydrochloric acid. Scientific Reports 2016, 6, 19890; Poolman, J. M.; Maity, C.; Boekhoven, J.; Mee, L. v. d.; Sage, V. A. A. 1; Groenewold, G. J. M.; Kasteren, S. I. v.; Versluis, F.; Esch, J. H. v.; Eelkema, R. A toolbox for controlling the properties and functionalisation of hydrazone-based supramolecular hydrogels. J. Mater. Chem. B 2016, 4, 852-859; Cougnon, F. B. L.; Caprice, K.; Pupier, M.; Bauzá, A.; Frontera, A. A Strategy to Synthesize Molecular Knots and Links Using the Hydrophobic Effect. J. Am. Chem. Soc. 2018, 140, 12442-12450). The reaction was refluxed for 6 h. After cooling down, the product was precipitated and filtered off. The product was dried in vacuum after washing with water, as a white solid (80%).

Heptanoic acid hydrazide: ¹H NMR (500 MHz, DMSO-d₆) δ=8.88 (s, 1H), 4.03 (s, 1H), 1.96 (t, J=7.4, 2H), 1.43 (p, J=7.2, 2H), 1.20 (dtd, J=12.6, 8.6, 8.0, 4.6, 6H), 0.82 (t, J=6.9, 3H); ¹³C NMR (126 MHz, DMSO-d₆) δ=172.20, 33.95, 31.54, 28.86, 25.72, 22.54, 14.46; HR-ESI-MS calculated for C₇H₁₆N₂₀[M+H]⁺: 145.21, found 145.18.

Flow chemistry for synthesis of hexyl isocyanate: The flow system was set up as described in FIG. 2.

Solution 1: sodium nitrite (1.37 g, 0.02 mol) was distilled into 100 mL deionized water. The concentration was 0.2M

Solution 2: pure toluene

Solution 3: heptanoic acid hydrazide (1.44 g, 0.01 mol) was dissolved in 100 mL deionized water and 0.8 mL conc. HCl. The concentration was 0.1M.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing hexyl isocyanate product was placed in an Erlenmeyer flask. After removing solvent, hexyl isocyanate was collected as a colorless liquid and stored at a temperature of 4° C. This system provides hexyl isocyanate at a rate of 500 mg h⁻¹.

Hexyl isocyanate: ¹H NMR (500 MHz, Chloroform-d) δ=7.29 (t, J=7.6, 2H), 7.20 (dd, J=13.1, 7.1, 3H), 3.30 (t, J=6.8, 2H), 2.39 (s, 3H), 1.64 (d, J=14.8, 1H), 1.46-1.28 (m, 9H), 0.94 (q, J=6.3, 5.9, 4H); ¹³C NMR (126 MHz, Chloroform-d) δ=137.98, 129.17, 128.36, 125.44, 122.10, 43.10, 31.67, 31.53, 31.41, 31.28, 29.04, 26.35, 25.64, 22.81, 22.66, 21.54, 14.24, 14.14, 14.08. HR-ESI-MS calculated for C₇H₁₃NO [M+H]⁺: 128.18, found 128.14

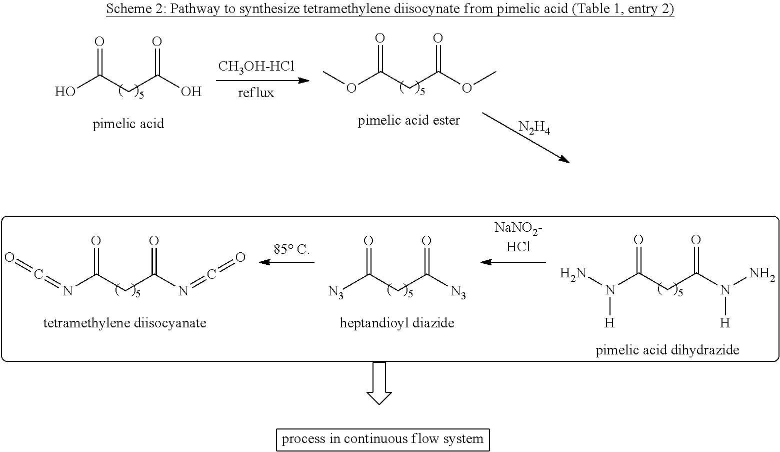

Synthesis of pimelic acid ester: Pimelic acid (20 g, 0.13 mol) was added to a 250 mL one-neck round-bottom flask, then 100 mL methanolic HCl 1M was poured into this flask. The reaction was reflux for 2 h⁵. Once finishing reaction, 100 mL hexane was added to the flask. Two layers were divided by separating funnel. The organic layer was dried over anhydrous Na₂SO₄ and evaporated to afford pimelic acid ester as a colorless oil with the yield of 82%.

Pimelic acid ester: ¹H NMR (500 MHz, Chloroform-d) δ=3.62 (s, 3H), 2.27 (t, J=7.6, 2H), 1.60 (p, J=7.4, 2H), 1.36-1.26 (m, 1H).

Synthesis of pimelic acid dihydrazide: 16.5 mL N₂H4 (0.5 mol) was added slowly to 80 mL methanol contain 10 g pimelic acid ester (0.05 mol) in 150 mL one-neck round-bottom flask (Al-Amiery, A. A.; Kassim, F. A. B.; Kadhum, A. A. H.; Mohamad, A. B. Synthesis and characterization of a novel eco-friendly corrosion inhibition for mild steel in 1M hydrochloric acid. Scientific Reports 2016, 6, 19890; Poolman, J. M.; Maity, C.; Boekhoven, J.; Mee, L. v. d.; Sage, V. A. A. 1; Groenewold, G. J. M.; Kasteren, S. I. v.; Versluis, F.; Esch, J. H. v.; Eelkema, R. A toolbox for controlling the properties and functionalisation of hydrazone-based supramolecular hydrogels. J. Mater. Chem. B 2016, 4, 852-859; Cougnon, F. B. L.; Caprice, K.; Pupier, M.; Bauzá, A.; Frontera, A. A Strategy to Synthesize Molecular Knots and Links Using the Hydrophobic Effect. J. Am. Chem. Soc. 2018, 140, 12442-12450). The reaction was refluxed for 6 h. After cooling down, pimelic acid dihydrazide (88% in yield) is collected as a white powder after filtration and washing with methanol.

Pimelic acid dihydrazide: ¹H NMR (500 MHz, DMSO-d₆) δ=8.93 (s, 1H), 4.12 (s, 2H), 1.95 (t, J=7.5, 2H), 1.42 (p, J=7.5, 2H), 1.14 (p, J=7.9, 1H); ¹³C NMR (126 MHz, DMSO-d₆) δ=172.08, 33.84, 28.83, 25.54; HR-ESI-MS calculated for C₇H₁₆N₄O₂[M+H]⁺: 189.23, found 189.13

Synthesis of tetramethylene diisocyanate: The flow system was set up as described in FIG. 2.

Solution 1: sodium nitrite (1.37 g, 0.02 mol) was distilled into 100 mL deionized water. The concentration was 0.2M

Solution 2: pure toluene

Solution 3: pimelic acid dihydrazide (1.88 g, 0.01 mol) was dissolved in 100 mL deionized water and 0.8 mL conc. HCl. The concentration was 0.1M.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing tetramethylene diisocyanate product was placed in an Erlenmeyer flask. After removing solvent, tetramethylene diisocyanate was collected as a colorless liquid and stored at a temperature of 4° C. This system provides tetramethylene diisocyanate at a rate of 500 mg h⁻¹.

Tetramethylene diisocyanate: ¹H NMR (500 MHz, Chloroform-d) 6=3.33 (t, J=6.6, 2H), 1.68-1.61 (m, 1H), 1.48 (tt, J=10.4, 6.0, 1H); ¹³C NMR (126 MHz, Chloroform-d) 6=138.00, 129.16, 128.35, 125.42, 42.87, 30.70, 23.75, 21.56; HR-ESI-MS calculated for C₇H₁₀N₂O₂[M+H]⁺: 155.17, found 155.05.

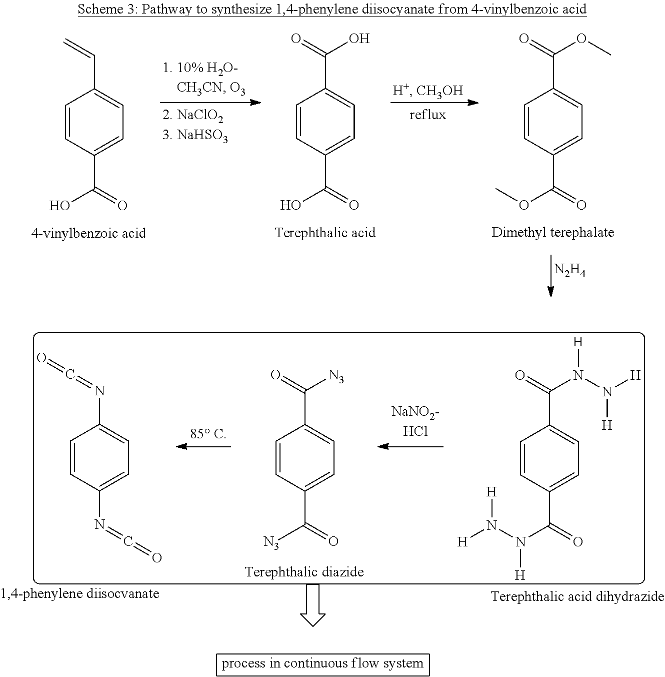

Synthesis of terephthalic acid (Cochran, B. M. One-Pot Oxidative Cleavage of Olefins to Synthesize Carboxylic Acids by a Telescoped Ozonolysis-Oxidation Process. Synlett 2016, 27, 245-248): A solution of 4-vinylbenzoic acid (10 g, 0.06 mol, 1 equiv) dissolved in 160 mL acetonitrile and 16 mL H₂O was added to a 500 mL round-bottom flask. The solution was cooled to 0° C. in an ice bath and exposed to ozone. Complete reaction can be verified by thin-layer chromatography (TLC). Once the ozonolysis completed, a 135 mL aqueous solution of sodium chlorite 2M (24.41 g, 0.26 mol, 4 equiv) was slowly added to the cold reaction. The internal reaction temperature should be under 15° C. during the portion-wise adding of NaClO₂. The reaction mixture turned yellow after adding sodium chlorite solution. The mixture was then stirred overnight. Next, 135 mL aqueous solution of sodium bisulfite 2M (28.09 g, 0.26 mol, 4 equiv) was dropped wise to the reaction mixture maintaining the temperature under 15° C. The solution turned clear after the solution of sodium bisulfite was completely added. The mixture was stirred for 10 minutes. A white precipitate was filtered and washed with water (100 mL) to afford a white solid (85%).

Synthesis of dimethyl terephthalate: Terephthalic acid (10 g, 0.06 mol), methanol (110 mL) and conc H₂SO₄ (2.7 mL) were heated under reflux for 4 h (Datoussaida, Y.; Othmana, A. A.; Kirsch, G. Synthesis and Antibacterial Activity of some 5,5′-(1,4-phenylene)-bis-1,3,4-Oxadiazole and bis-1,2,4-Triazole Derivatives as Precursors of New S-Nucleosides. S. Afr. J. Chem. 2012, 65, 30-35). Once the reaction complete, dichloromethane (100 mL) and water (100 mL) were added to the mixture. After separation, the organic phase was dried over anhydrous Na₂SO₄ and evaporated to afford a white solid (85%).

Dimethyl terephthalate: ¹H NMR (500 MHz, Chloroform-d) δ=8.10 (s, 3H), 3.95 (s, OH), 3.94 (s, 6H); ¹³C NMR (126 MHz, Chloroform-d) δ=166.43, 133.99, 129.66, 52.56.

Synthesis of terephthalic acid dihydrazide: Dimethyl terephthalate (6 g, 0.03 mol), methanol (60 mL) and hydrazine (9.6 mL, 0.3 mol) were heated under reflux for 6 h (Datoussaida, Y.; Othmana, A. A.; Kirsch, G. Synthesis and Antibacterial Activity of some 5,5′-(1,4-phenylene)-bis-1,3,4-Oxadiazole and bis-1,2,4-Triazole Derivatives as Precursors of New S-Nucleosides. S. Afr. J. Chem. 2012, 65, 30-35). The precipitated terephthalic acid dihydrazide was filtered and washed with methanol to afford a yellowish-white solid (85%).

Terephthalic acid dihydrazide: ¹H NMR (500 MHz, DMSO-d₆) δ=9.86 (s, 3H), 7.98 (d, J=8.1, 1H), 7.89 (d, J=8.2, 1H), 7.82 (s, 3H), 3.83 (s, 1H); ¹³C NMR (126 MHz, DMSO-d₆) δ=165.74, 135.98, 129.72, 127.91, 127.51; HR-ESI-MS calculated for C₈H₁₀N₄O₂[M+H]⁺: 195.19, found 195.23

### Synthesis of 1,4-phenylene diisocyanate

Solution 1: sodium nitrite (1.37 g, 0.02 mol) was distilled into 100 mL deionized water. The concentration was 0.2M

Solution 2: a mixture of toluene and acetonitrile (50/50 v/v)

Solution 3: terephthalic acid dihydrazide (1.88 g, 0.01 mol) was dissolved in 100 mL deionized water and 0.8 mL conc. HCl. The concentration was 0.1M.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscille liquids, toluene and water. After complete separation, water exited on one side and was collected in a waste Erlenrneyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing 1,4-phenylene diisocyanate product was placed in an Erlenmeyer flask. After removing solvent, 1,4-phenylene diisocyanate was collected as an orange solid and stored at a temperature of 4° C. This system provides 1,4-phenylene diisocyanate at a rate of 500 mg h⁻¹.

1,4-phenylene diisocyanate: ¹H NMR (500 MHz, Chloroform-d) δ=8.15-8.06 (m, 2H), 8.10 (s, 6H), 8.00 (t, J=7.7, 1H), 7.26 (t, J=7.4, 5H), 7.21-7.11 (m, 9H), 3.96 (s, 2H), 2.94 (s, 1H), 2.36 (s, 8H), 1.98 (s, 0H), 1.28 (q, J=9.1, 7.3, 1H), 0.90 (q, J=6.6, 1H); ¹³C NMR (126 MHz, Chloroform-d) δ=171.76, 137.98, 135.37, 134.30, 131.19, 129.91, 129.72, 129.52, 129.16, 128.35, 127.86, 125.43, 125.13, 124.82, 52.67, 21.56; HR-ESI-MS calculated for C₈H₄N₂O₂[M+H]⁺: 161.13, found 161.02.

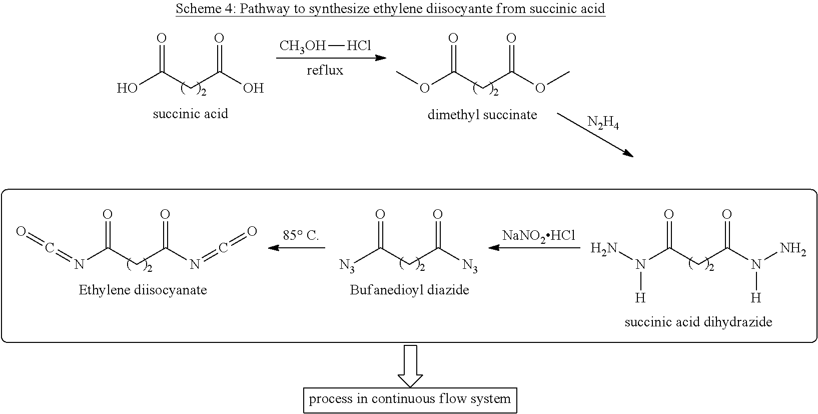

Synthesis of dimethyl succinate: Succinic acid (5 g, 0.04 mol) was added to a 100 mL one-neck round-bottom flask, then 50 mL methanolic HCl 1M was poured into this flask. The reaction was reflux for 2 h. Once finishing reaction, 50 mL hexane was added to the flask. Two layers were divided by separating funnel. The organic layer was dried over anhydrous Na₂SO₄ and evaporated to afford dimethyl succinate as a colorless oil with the yield of 78%.

Dimethyl succinate: ¹H NMR (500 MHz, Chloroform-d) δ=3.64 (s, 2H), 2.58 (s, 1H).

Synthesis of succinic acid dihydrazide: 6.5 mL N₂H₄ (0.2 mol) was added slowly to 30 mL methanol contain 3 g dimethyl succinate (0.02 mol) in 100 mL one-neck round-bottom flask. The reaction was refluxed for 6 h. After cooling down, succinic acid dihydrazide (79% in yield) is collected as a white powder after filtration and washing with methanol.

¹H NMR (500 MHz, DMSO-d₆) δ=8.95 (s, 2H), 4.10 (s, 4H), 2.21 (s, 3H); ¹³C NMR (126 MHz, DMSO-d₆) δ=171.32, 29.46; HR-ESI-MS calculated for C₄H₁₀N₄O₂[M+H]⁺: 147.15, found 147.06.

Synthesis of ethylene diisocyanate: The flow system was set up as described in FIG. 2.

Solution 1: sodium nitrite (0.55 g, 0.008 mol) was distilled into 100 mL deionized water. The concentration was 0.2M

Solution 2: pure toluene

Solution 3: succinic acid hydrazide (0.58 g, 0.004 mol) was dissolved in 40 mL deionized water and 1.3 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, ethylene diisocyanate was collected as a colorless liquid (70%) and stored at a temperature of 4° C.

¹H NMR (500 MHz, Chloroform-d) δ=7.31 (t, J=7.5, 2H), 7.22 (d, J=6.8, 2H), 3.47 (s, 6H), 2.41 (s, 3H); ¹³C NMR (126 MHz, Chloroform-d) δ=138.06, 129.22, 129.17, 128.40, 125.47, 125.42, 123.99, 44.01, 21.60, 21.55, 21.50, 21.45; HR-ESI-MS calculated for C₄H₄N₂O₂ [M+MeOH+H]⁺: 145.04, found 145.13.

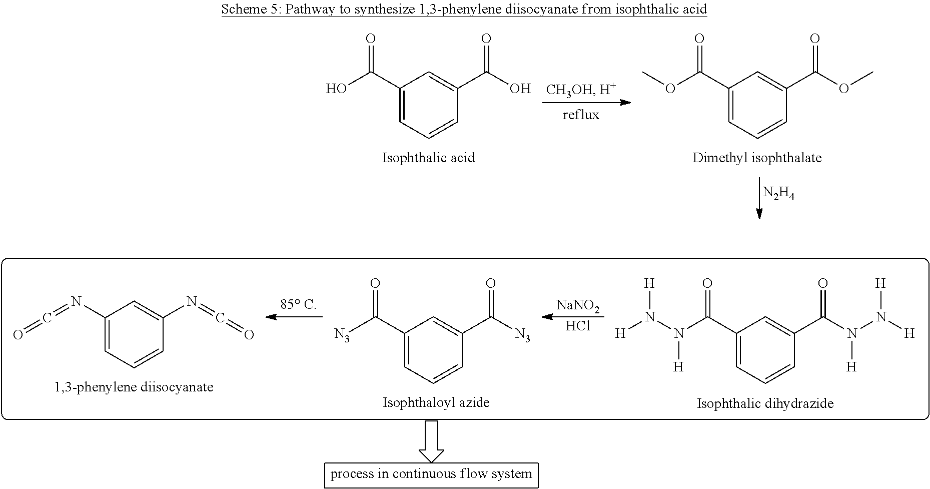

Synthesis of dimethyl isophthalate: Isophthalic acid (5.89 g, 0.035 mol) was added to a 100 mL one-neck round-bottom flask, then 40 mL methanolic HCl 1M was poured into this flask. The reaction was reflux for 2 h. Once finishing reaction, 100 mL hexane was added to the flask. Two layers were divided by separating funnel. The organic layer was dried over anhydrous Na₂SO₄ and evaporated to afford desired product as a white powder with the yield of 80%.

Dimethyl isophthalate: ¹H NMR (500 MHz, Chloroform-d) δ=8.68 (s, 1H), 8.25 (s, 1H), 8.23 (dd, J=8.0, 1.7, 2H), 7.59-7.50 (m, 1H), 3.95 (s, 8H); ¹³C NMR (126 MHz, Chloroform-d) δ=166.38, 133.92, 130.83, 130.69, 128.75, 52.49.

Synthesis of Isophthalic dihydrazide: 8.0 mL N₂H₄ (0.25 mol) was added slowly to 50 mL methanol contain 5 g dimethyl isophthalate (0.025 mol) in 150 mL one-neck round-bottom flask. The reaction was refluxed for 6 h. After cooling down, isophthalic dihydrazide (83% in yield) is collected as a white powder after filtration and washing with methanol.

Isophthalic dihydrazide: ¹H NMR (500 MHz, DMSO-d₆) δ=9.80 (s, 2H), 8.22 (s, 1H), 7.88 (d, J=7.5, 2H), 7.49 (t, J=7.7, 1H), 4.51 (s, 4H); ¹³C NMR (126 MHz, DMSO-d₆) δ=166.01, 134.06, 129.85, 128.99, 126.57; HR-ESI-MS calculated for C₈H₁₀N₄O₂[M+H]⁺: 195.19, found 195.18

Synthesis of 1,3-phenylene diisocyanate: The flow system was set up as described in FIG. 2.

Solution 1: sodium nitrite (0.34 g, 0.005 mol) was distilled into 50 mL deionized water.

Solution 2: pure toluene

Solution 3: isophthalic dihydrazide (0.48 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, 1,3-phenylene diisocyanate was collected as a pale yellow powder (65%) and stored at a temperature of 4° C.

1,3-phenylene diisocyanate: ¹H NMR (500 MHz, Chloroform-d) δ=7.55-7.46 (m, 3H), 7.41 (d, J=7.6, 4H), 2.59 (s, 5H); ¹³C NMR (126 MHz, Chloroform-d) δ=138.13, 134.93, 130.67, 130.54, 130.18, 129.39, 128.59, 126.88, 125.74, 125.68, 122.49, 21.73; HR-ESI-MS calculated for C₈H₄N₂O₂[M+H]⁺: 161.13, found 161.23.

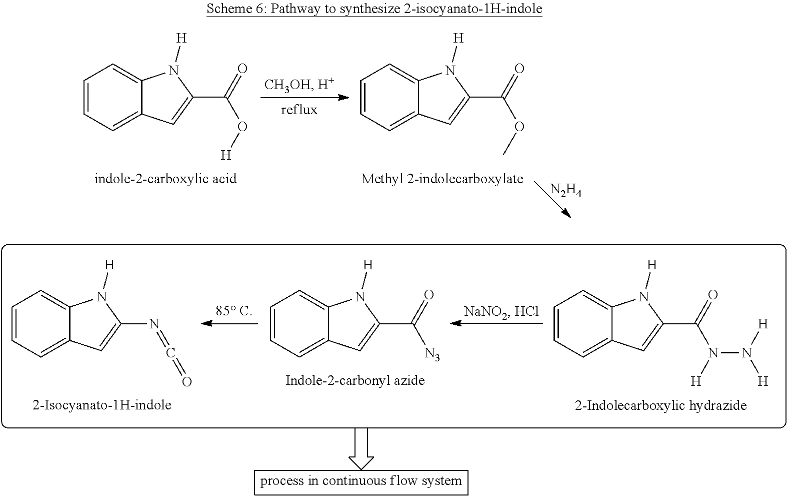

Synthesis of methyl 2-indolecarboxylate: 4 mL N₂H₄ (0.125 mol) was added slowly to 25 mL methanol contain 2.2 g methyl-2-indolecarboxylate (0.0125 mol) in 100 mL one-neck round-bottom flask. The reaction was refluxed for 6 h. After cooling down, 2-indolecarboxylic hydrazide (75% in yield) is collected as a white powder after filtration and washing with methanol.

2-Indolecarboxylic hydrazide: ¹H NMR (500 MHz, DMSO-d₆) δ=11.53 (s, 1H), 9.77 (s, 1H), 7.55 (d, J=7.9, 1H), 7.40 (d, J=8.2, 1H), 7.13 (t, J=7.6, 1H), 7.04 (s, 1H), 6.99 (t, J=7.4, 1H); ¹³C NMR (126 MHz, DMSO-d₆) δ=161.79, 136.85, 130.88, 127.62, 123.79, 121.97, 120.34, 112.81, 102.55; HR-ESI-MS calculated for C₉H₉N₃O [M+H]⁺: 176.19, found 176.15.

Synthesis of 2-Isocyanato-1H-indole: The flow system was set up as described in FIG. 2.

Solution 1: sodium nitrite (0.37 g, 0.005 mol) was distilled into 100 mL deionized water.

Solution 2: pure toluene

Solution 3: 2-indolecarboxylic hydrazide (0.44 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a dark brown liquid (76%) and stored at a temperature of 4° C.

2-Isocyanato-1H-indole: ¹H NMR (500 MHz, Chloroform-d) δ=7.24 (t, J=7.4, 1H), 7.15 (dd, J=13.7, 7.2, 2H), 2.34 (s, 2H), 1.92 (s, 6H), 1.34-1.24 (m, 1H), 0.92-0.83 (m, 1H); ¹³C NMR (126 MHz, Chloroform-d) δ=137.95, 129.14, 128.35, 126.58, 125.42, 123.04, 121.28, 116.75, 112.46, 110.83, 53.75, 31.70, 22.76, 21.46, 14.19, 1.74; HR-ESI-MS calculated for C₉H₆N₂O [M+H]⁺: 159.16, found 159.22.

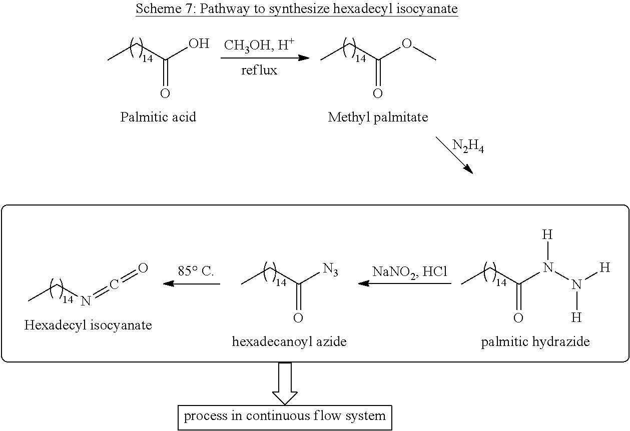

Synthesis of methyl palmitate: Palmitic acid (6.36 g, 0.025 mol) was added to a 250 mL one-neck round-bottom flask, then 50 mL methanolic HCl 1M was poured into this flask. The reaction was reflux for 2 h. Once finishing reaction, 100 mL hexane was added to the flask. Two layers were divided by separating funnel. The organic layer was dried over anhydrous Na₂SO₄ and evaporated to afford desired product as a pale yellow oil with the yield of 80%.

Methyl palmitate: ¹H NMR (500 MHz, Chloroform-d) δ=3.65 (s, 1H), 2.29 (t, J=7.6, 1H), 1.64-1.57 (m, 1H), 1.28 (dt, J=13.9, 6.4, 5H), 1.24 (s, 7H), 0.90-0.82 (m, 2H); ¹³C NMR (126 MHz, Chloroform-d) δ=174.44, 51.51, 34.20, 32.02, 31.69, 29.78, 29.74, 29.69, 29.55, 29.46, 29.36, 29.25, 27.31, 25.05, 22.79, 22.75, 14.20

Synthesis of palmitic hydrazide: 2.5 mL N₂H₄ (0.073 mol) was added slowly to 40 mL methanol contain 2 g methyl palmitate (0.0073 mol) in 100 mL one-neck round-bottom flask. The reaction was refluxed for 6 h. After cooling down, desired product (68% in yield) is collected as a white powder after filtration and washing with methanol.

Palmitic hydrazide: ¹H NMR (500 MHz, DMSO-d₆) δ=8.89 (s, 1H), 2.68 (s, 1H), 1.95 (t, J=7.4, 2H), 1.42 (s, 3H), 1.19 (s, 22H), 1.18 (s, 2H), 0.81 (t, J=6.7, 3H); HR-ESI-MS calculated for C₁₆H₃₄N₂O [M+H]⁺: 271.45, found 271.40.

Synthesis of hexadecyl isocyanate: The flow system was set up as described in FIG. 2.

Solution 1: sodium nitrite (0.35 g, 0.005 mol) was distilled into 50 mL deionized water.

Solution 2: pure toluene

Solution 3: palmitic hydrazide (0.68 g, 0.0025 mol) was dissolved in 50 mL deionized water and 0.8 mL aqueous HCl 6N.

All solutions were pumped with a flow rate of 1 mL mint. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a pale yellow liquid (72%) and stored at a temperature of 4° C.

Hexadecyl isocyanate: ¹H NMR (500 MHz, Chloroform-d) δ=7.45 (t, J=7.5, 5H), 7.37 (dd, J=7.9, 3.0, 8H), 2.55 (s, 9H), 1.96 (s, 2H), 1.13 (t, J=6.8, 1H). ¹³C NMR (126 MHz, Chloroform-d) δ=138.11, 129.35, 128.54, 125.63, 116.61, 31.96, 30.08, 30.04, 29.75, 23.02, 21.69, 14.44, 1.70. HR-ESI-MS calculated for C₁₆H₃₁NO [M+MeOH+H]⁺: 286.49, found 286.26

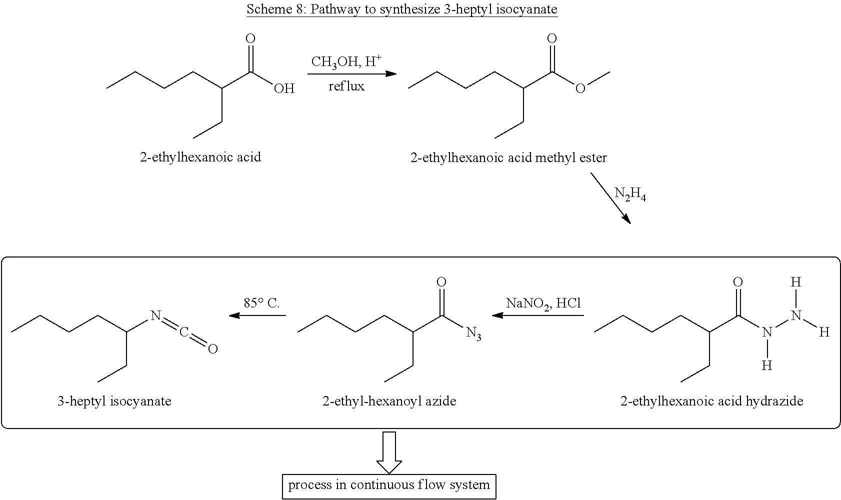

Synthesis of 2-ethylhexanoic acid methyl ester: 2-ethylhexanoic acid (5.85 g, 0.04 mol) was added to a 100 mL one-neck round-bottom flask, then 50 mL methanolic HCl 1M was poured into this flask. The reaction was reflux for 2 h. Once finishing reaction, 100 mL hexane was added to the flask. Two layers were divided by separating funnel. The organic layer was dried over anhydrous Na₂SO₄ and evaporated to afford desired product as a colorless oil with the yield of 85%.

2-Ethylhexanoic acid methyl ester: ¹H NMR (500 MHz, Chloroform-d) δ=3.66 (s, 2H), 2.25 (tt, J=8.7, 5.3, 1H), 1.66-1.38 (m, 3H), 1.35-1.15 (m, 4H), 0.86 (t, J=7.3, 5H). ¹³C NMR (126 MHz, Chloroform-d) δ=177.05, 51.35, 47.36, 31.90, 31.68, 29.74, 25.57, 25.35, 22.71, 14.18, 14.01, 11.92.

Synthesis of 2-ethylhexanoic acid hydrazide: 4 mL N₂H₄ (0.5 mol) was added slowly to 40 mL methanol contain 2 g 2-ethylhexanoic acid methyl ester (0.012 mol) in 150 mL one-neck round-bottom flask. The reaction was refluxed for 6 h. After cooling down, a desired product (68% in yield) is collected as a white powder after filtration and washing with methanol.

2-ethylhexanoic acid hydrazide: ¹H NMR (500 MHz, DMSO-d₆) δ=3.55 (s, 3H), 2.20 (tt, J=8.6, 5.5, 1H), 1.52-1.42 (m, 2H), 1.45-1.37 (m, 2H), 1.40-1.31 (m, 1H), 1.29-1.04 (m, 4H), 0.83-0.69 (m, 8H). ¹³C NMR (126 MHz, DMSO-d₆) δ=176.37, 51.62, 46.93, 46.10, 32.41, 31.78, 29.78, 29.59, 26.03, 25.50, 22.69, 22.57, 14.38, 14.26, 12.36, 12.09. HR-ESI-MS calculated for C₈H₁₈N₂O [M+H]⁺: 159.24, found 159.23.

Synthesis of 3-heptyl isocyanate: The flow system was set up as described in FIG. 2.

Solution 1: sodium nitrite (0.34 g, 0.005 mol) was distilled into 50 mL deionized water.

Solution 2: pure toluene

Solution 3: 2-ethylhexanoic acid hydrazide (0.39 g, 0.0025 mol) was dissolved in 50 mL deionized water and 0.8 mL aqueous HCl 6N.

All solutions were pumped with a flow rate of 1 mL min⁻¹, The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a colorless liquid (71%) and stored at a temperature of 4° C.

3-Heptyl isocyanate: ¹H NMR (500 MHz, Chloroform-d) δ=7.31 (t, J=7.5, 6H), 7.27-7.18 (m, 5H), 3.40 (ddd, J=12.9, 8.1, 4.7, 1H), 2.41 (s, 9H), 1.70-1.45 (m, 5H), 1.45-1.29 (m, 4H), 1.07-0.93 (m, 7H). ¹³C NMR (126 MHz, Chloroform-d) δ=138.02, 129.21, 128.40, 125.47, 57.99, 36.32, 30.03, 28.48, 22.52, 21.60, 14.14, 10.65. HR-ESI-MS calculated for C₈H₁₅NO [M+3H]⁺: 144.21, found 144.20.

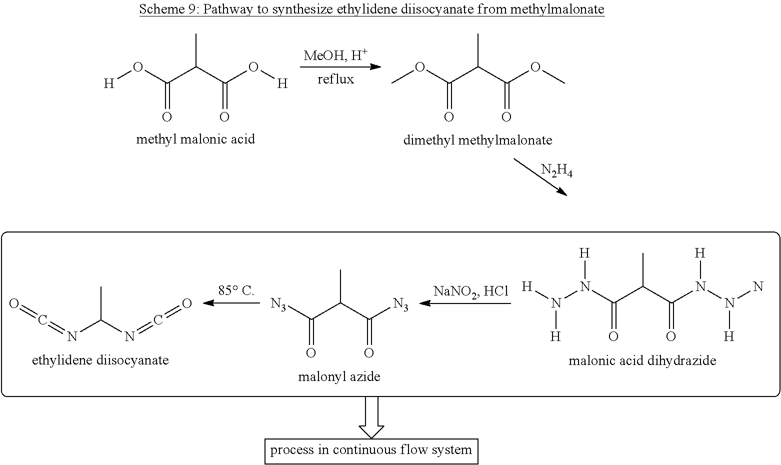

Synthesis of dimethyl methylmalonate: Methyl malonic acid (0.5 g, 0.0042 mol) was added to a 50 mL one-neck round-bottom flask, then 10 mL methanolic HCl 1M was poured into this flask. The reaction was reflux for 2 h. Once finishing reaction, 20 mL hexane was added to the flask. Two layers were divided by separating funnel. The organic layer was dried over anhydrous Na₂SO₄ and evaporated to afford desired product as a colorless oil with the yield of 85%.

Dimethyl methylmalonate: ¹H NMR (500 MHz, DMSO-d₆) δ=3.62 (s, 7H), 3.58 (q, J=7.3, 1H), 1.24 (d, J=7.1, 4H). ¹³C NMR (126 MHz, DMSO-d₆) δ=170.76, 52.85, 45.56, 14.01.

Synthesis of malonic acid dihydrazide: 0.8 mL N₂H₄ (0.023 mol) was added slowly to 5 mL methanol contain 0.35 g dimethyl methylmalonate (0.0023 mol) in 20 mL one-neck round-bottom flask. The reaction was refluxed for 6 h. After cooling down, desired product (57% in yield) is collected as a white powder after filtration and washing with methanol.

Malonic acid dihydrazide: ¹H NMR (500 MHz, DMSO-d₆) δ=8.84 (s, 2H), 4.18 (s, 4H), 3.00 (q, J=7.1, 1H), 1.13 (d, J=7.3, 3H). ¹³C NMR (126 MHz, DMSO-d₆) δ=170.04, 44.19, 14.89. HR-ESI-MS calculated for C₄H₁₀N₄O₂[M+H]⁺: 147.15, found 147.17.

Synthesis of ethylidene diisocyanate: The flow system was set up as described in FIG. 2.

Solution 1: sodium nitrite (0.34 g, 0.005 mol) was distilled into 50 mL deionized water.

Solution 2: pure toluene

Solution 3: malonic acid dihydrazide (0.37 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s, The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a pale yellow liquid (71%) and stored at a temperature of 4° C.

Ethylidene diisocyanate: ¹H NMR (500 MHz, Chloroform-d) δ=7.29 (t, J=7.6, 34H), 7.20 (dd, J=12.1, 7.3, 49H), 3.51 (s, 1H), 2.39 (s, 52H), 2.04 (d, J=38.5, 2H), 1.42-1.18 (m, 1H). ¹³C NMR (126 MHz, Chloroform-d) δ=138.02, 129.19, 128.38, 125.45, 21.60. HR-ESI-MS calculated for C₄H₄N₂O₂[M+H]⁺: 113.09, found 113.03

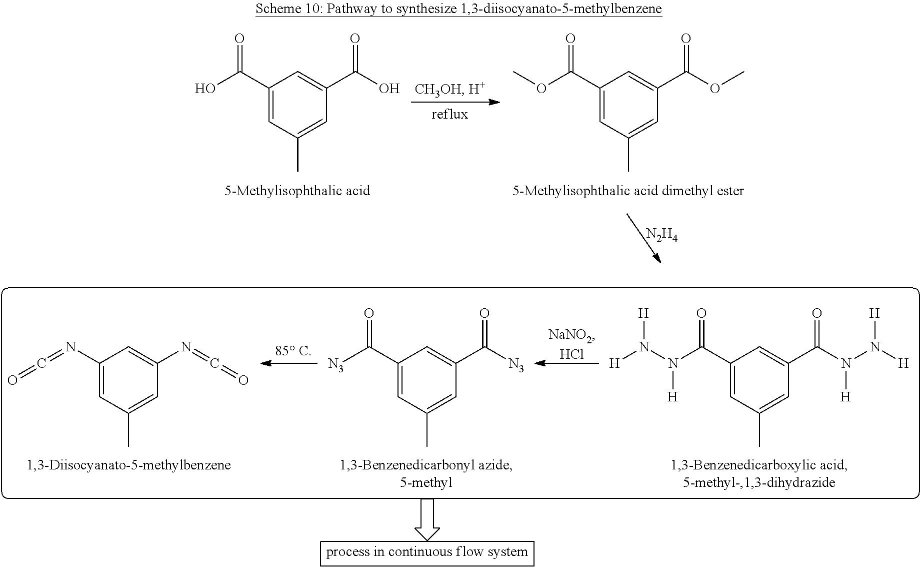

Synthesis of 5-methylisophthalic acid dimethyl ester: 5-Methylisophthalic acid (0.4 g, 0.0022 mol) was added to a 50 mL one-neck round-bottom flask, then 5 mL methanolic HCl 1M was poured into this flask. The reaction was reflux for 2 h. Once finishing reaction, 20 mL hexane was added to the flask. Two layers were divided by separating funnel. The organic layer was dried over anhydrous Na₂SO₄ and evaporated to afford desired product as a white powder with the yield of 68%.

5-methylisophthalic acid dimethyl ester: ¹H NMR (500 MHz, DMSO-d₆) δ=8.21 (s, 1H), 7.96 (s, 2H), 3.83 (s, 9H), 2.38 (s, 4H). ¹³C NMR (126 MHz, DMSO-d₆) δ=166.04, 139.87, 134.53, 130.61, 127.39, 52.94, 21.06.

Synthesis of 1,3-Benzenedicarboxylic acid, 5-methyl-,1,3-dihydrazide: 0.6 mL N₂H₄ (0.015 mol) was added slowly to 5 mL methanol contain 0.3 g 5-methylisophthalic acid dimethyl ester (0.0014 mol) in 20 mL one-neck round-bottom flask. The reaction was refluxed for 6 h. After cooling down, 1,3-Benzenedicarboxylic acid, 5-methyl-,1,3-dihydrazide (74% in yield) is collected as a white powder after filtration and washing with methanol.

1,3-Benzenedicarboxylic acid, 5-methyl-,1,3-dihydrazide: ¹H NMR (500 MHz, DMSO-d₆) δ=9.71 (s, 2H), 8.01 (s, 1H), 7.71 (s, 2H), 4.48 (s, 4H), 3.84 (s, 0H), 2.34 (s, 3H). ¹³C NMR (126 MHz, DMSO-d₆) δ=166.13, 138.37, 134.06, 130.48, 123.72, 21.44. HR-ESI-MS calculated for C₉H₁₂N₄O₂[M+H]⁺: 209.22, found 209.11

Synthesis of 1,3-Diisocyanato-5-methylbenzene: The flow system was set up as described in FIG. 2.

Solution 1: sodium nitrite (0.34 g, 0.005 mol) was distilled into 50 mL deionized water.

Solution 2: pure toluene

Solution 3: 1,3-Benzenedicarboxylic acid, 5-methyl-,1,3-dihydrazide (0.52 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a colorless liquid (60%) and stored at a temperature of 4° C.

1,3-Diisocyanato-5-methylbenzene: ¹H NMR (500 MHz, Chloroform-d) δ=8.48 (s, 1H), 8.11 (s, 3H), 7.70 (s, 2H), 7.58 (d, J=8.1, 0H), 7.57 (s, 2H), 7.29 (t, J=7.5, 19H), 7.22 (s, 12H), 7.23-7.14 (m, 19H), 6.76 (d, J=1.9, 2H), 6.64 (q, J=2.6, 1H), 3.96 (d, J=14.9, 1H), 3.81 (d, J=9.1, 1H), 2.49 (d, J=8.8, 1H), 2.49 (s, 6H), 2.40 (d, J=7.4, 40H), 2.32 (d, J=3.5, 4H), 1.30 (s, 1H). ¹³C NMR (126 MHz, Chloroform-d) δ=171.78, 140.52, 138.00, 135.44, 134.16, 131.43, 131.10, 129.18, 128.37, 127.87, 127.49, 125.45, 125.26, 123.17, 122.80, 118.09, 21.58, 21.22, 21.17. HR-ESI-MS calculated for C₉H₆N₂O₂[M+H]⁺: 175.16, found 175.15.

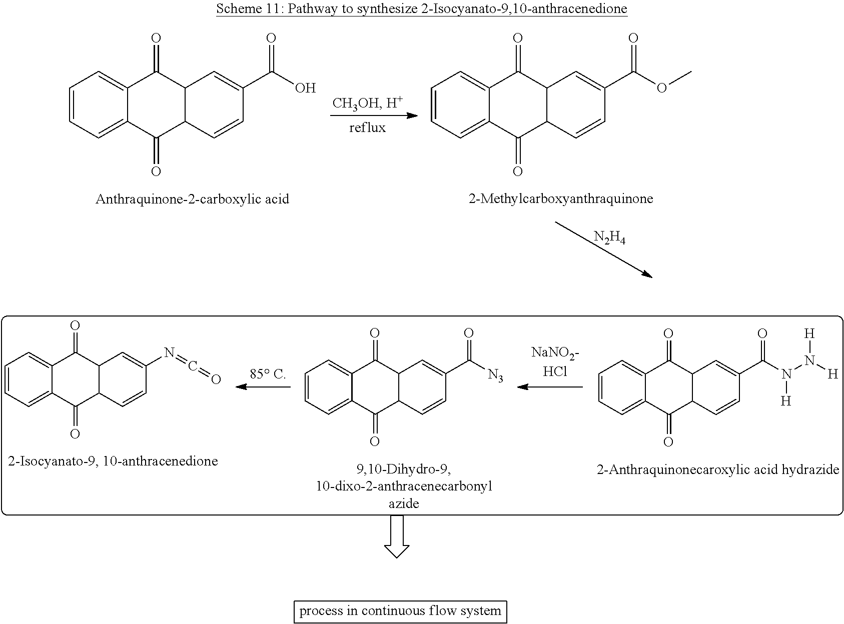 [SMILES: O=C(O)C1=CC2C(=O)c3ccccc3C(=O)C2C=C1 ; COC(=O)C1=CC2C(=O)c3ccccc3C(=O)C2C=C1 ; "N2H4" ; O=C=NC1=CC2C(=O)c3ccccc3C(=O)C2C=C1 ; [N-]=[N+]=NC(=O)C1=CC2C(=O)c3ccccc3C(=O)C2C=C1 ; NNC(=O)C1=CC2C(=O)c3ccccc3C(=O)C2C=C1]

Synthesis of 2-Methylcarboxyanthraquinone: Anthraquinone-2-carboxylic acid (0.3 g, 0.0012 mol) was added to a 250 mL one-neck round-bottom flask, then 4 mL methanolic HCl 1M was poured into this flask. The reaction was reflux for 2 h. Once finishing reaction, 10 mL hexane was added to the flask. Two layers were divided by separating funnel. The organic layer was dried over anhydrous Na₂SO₄ and evaporated to afford desired product as a white powder with the yield of 80%.

2-Methylcarboxyanthraquinone: ¹H NMR (500 MHz, Chloroform-d) δ=8.96 (s, 1H), 8.45 (d, J=9.2, 1H), 8.40 (d, J=7.8, 1H), 8.35 (s, 2H), 7.84 (s, 2H), 4.01 (d, J=6.9, 3H). ¹³C NMR (126 MHz, DMSO-d₆) δ=182.58, 182.42, 166.49, 136.19, 136.06, 135.29, 134.95, 133.65, 133.48, 127.88, 127.68, 127.39.

Synthesis of 2-Anthraquinonecarboxylic acid hydrazide: 0.28 mL N₂H₄ (0.0091 mol) was added slowly to 10 mL methanol contain 0.242 g 2-methylcarboxyanthraquinone (0.0009 mol) in 20 mL one-neck round-bottom flask. The reaction was refluxed for 6 h. After cooling down, desired product (77% in yield) is collected as a yellow powder after filtration and washing with methanol.

2-Anthraquinonecarboxylic acid hydrazide: ¹H NMR (500 MHz, DMSO-d₆) δ=10.23 (s, 1H), 8.62 (s, 2H), 8.27 (dd, J=23.0, 6.7, 3H), 8.23-8.13 (m, 8H), 7.91 (d, J=7.1, 6H). ¹³C NMR (126 MHz, DMSO-d₆) δ=61.63, 127.35, 127.99, 135.19, 153.94, 154.26. HR-ESI-MS calculated for C₁₅H₁₀N₂O₃[M+H]⁺: 267.25, found 267.25.

Synthesis of 2-Isocyanato-9,10-anthracenedione: The flow system was set up as described in FIG. 2.

Solution 1: sodium nitrite (0.34 g, 0.005 mol) was distilled into 50 mL deionized water.

Solution 2: pure toluene

Solution 3: 2-Anthraquinonecarboxylic acid hydrazide (0.67 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a colorless liquid (61%) and stored at a temperature of 4° C.

2-Isocyanato-9,10-anthracenedione: ¹H NMR (500 MHz, Chloroform-d) δ=7.31-7.25 (m, 4H), 7.19 (dd, J=12.4, 7.2, 6H), 3.50 (s, 0H), 2.38 (s, 5H), 1.62 (s, 1H). ¹³C NMR (126 MHz, Chloroform-d) δ=138.01, 129.18, 128.37, 125.44, 21.59. HR-ESI-MS calculated for C₁₅H₇NO₃[M+H]⁺: 250.22, found 250.20.

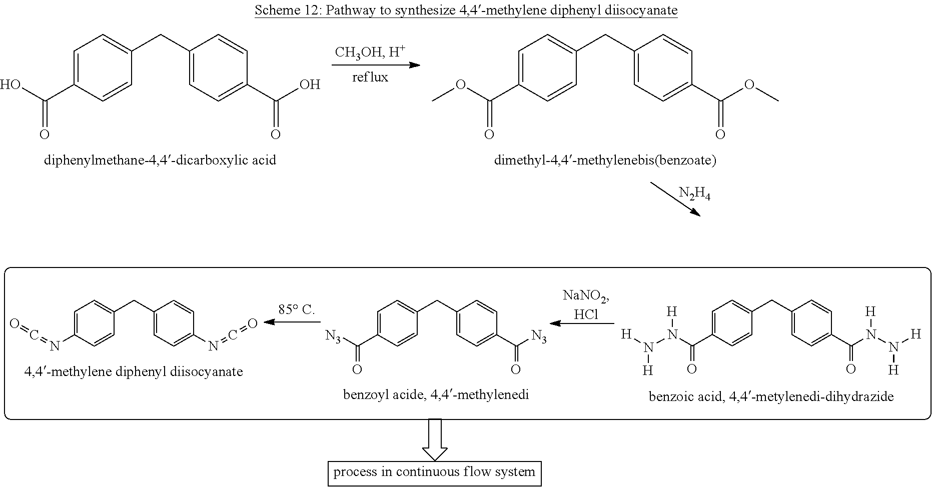

Synthesis of Dimethyl 4,4′-methylenebis(benzoate): Diphenylmethane-4,4′-dicarboxylic acid (0.41 g, 0.0016 mol) was added to a 20 mL one-neck round-bottom flask, then 5 mL methanolic HCl 1M was poured into this flask. The reaction was reflux for 2 h. Once finishing reaction, 10 mL hexane was added to the flask. Two layers were divided by separating funnel. The organic layer was dried over anhydrous Na₂SO₄ and evaporated to afford desired product as a white product with the yield of 78%.

Dimethyl 4,4′-methylenebis(benzoate): ¹H NMR (500 MHz, DMSO-d₆) δ=7.86 (d, J=8.0, 2H), 7.36 (d, J=8.0, 2H), 4.08 (s, 1H), 3.79 (s, 3H). ¹³C NMR (126 MHz, DMSO-d₆) δ=166.64, 146.69, 130.03, 129.74, 128.21, 52.60, 41.23.

Synthesis of Benzoic acid 4,4′-methylenedi-dihydrazide: 0.5 mL N₂H₄ (0.008 mol) was added slowly to 8 mL methanol contain 0.21 g dimethyl 4,4′-methylenebis(benzoate) (0.05 mol) in 20 mL one-neck round-bottom flask. The reaction was refluxed for 6 h. After cooling down, desired product (88% in yield) is collected as a light yellow powder after filtration and washing with methanol.

Benzoic acid 4,4′-methylenedi-dihydrazide: ¹H NMR (500 MHz, DMSO-d₆) δ=9.67 (s, 1H), 7.86 (d, J=7.8, 8H), 7.77 (d, J=7.7, 1H), 7.71 (d, J=7.7, 2H), 7.36 (d, J=7.9, 7H), 7.28 (d, J=7.6, 4H), 4.08 (s, 2H), 4.04 (d, J=3.8, 2H), 3.79 (s, 10H). ¹³C NMR (126 MHz, DMSO-d₆) δ=166.65, 146.70, 130.04, 128.20, 52.60, 41.22. HR-ESI-MS calculated for C₁₅H₁₆N₄O₂ [M+H]⁺: 285.31, found 285.22.

Synthesis of 4,4′-methylene diphenyl diisocyanate: The flow system was set up as described in FIG. 2.

Solution 1: sodium nitrite (0.34 g, 0.005 mol) was distilled into 50 mL deionized water.

Solution 2: pure toluene

Solution 3: Benzoic acid 4,4′-methylenedi-dihydrazide (0.71 g, 0.0025 mol) was dissolved in 50 mL deionized water and 0.8 mL aqueous HCl 6N.

All solutions were pumped with a flow rate of 1 mL min¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a white powder (58%) and stored at a temperature of 4° C.

4,4′-methylene diphenyl diisocyanate: ¹H NMR (500 MHz, Chloroform-d) δ=7.10 (d, J=8.3, 2H), 7.01 (d, J=7.9, 2H), 3.92 (s, 1H). ¹³C NMR (126 MHz, Chloroform-d) δ=138.43, 131.65, 130.04, 124.94, 124.74, 40.79. HR-ESI-MS calculated for C₁₅H₁₀N₂O₂ [M+2MeOH+Na]⁺: 337.33, found 337.19.

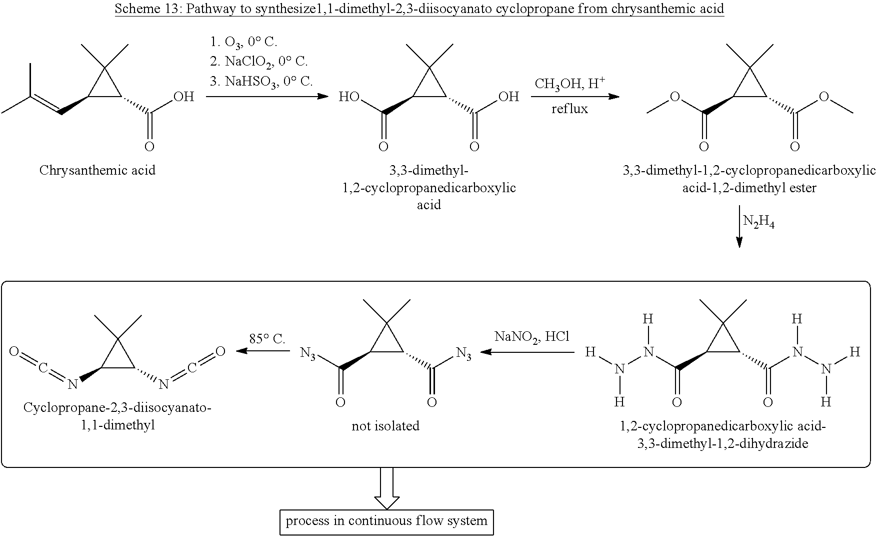

Synthesis of 3,3-dimethyl-1,2-cyclopropanedicarboxylic acid: The system for ozonolysis in batch mode. A solution of chrysanthemic acid (0.3 g, 0.0017 mol, 1 equiv) dissolved in 6 mL acetonitrile and 0.6 mL H₂O was added to a 20 mL round-bottom flask. The solution was cooled to 0° C. in an ice bath and exposed to ozone. Complete reaction can be verified by thin-layer chromatography (TLC). Once the ozonolysis was completed, a 3.6 mL 2 M aqueous solution of NaClO₂ (0.8 g, 0.0071 mol, 4 equiv) was slowly added to the cold reaction. The internal reaction temperature should be under 15° C. during the portion-wise adding of NaClO₂. The reaction mixture turned yellow after adding NaClO₂ solution. The mixture was then stirred overnight. Next, 3.9 mL 2M aqueous solution of sodium bisulfite (0.81 g, 0.0078 mol, 4 equiv) was added dropwise to the reaction mixture maintaining the temperature under 15° C. The solution turned clear after the solution of sodium bisulfite was completely added. The mixture was stirred for 10 min. Ethyl acetate (15 mL) was poured into the mixture, and the two layers were separated. The organic phase was collected, and solvent removed to provide a white slurry, which was a mixture of 3,3-dimethyl-1,2-cyclopropanedicarboxylic acid and isobutyric acid. Hot water and hexane were used to separate 3,3-dimethyl-1,2-cyclopropanedicarboxylic acid and isobutyric acid. On cooling the aqueous phase, 3,3-dimethyl-1,2-cyclopropanedicarboxylic acid was formed as a white crystal after being filtered, cleaned several times with cold water and dried. The hexane layer containing isobutyric acid was then dried over Na₂SO₄, filtered, and concentrated to obtain an colorless liquid. The yields of 3,3-dimethyl-1,2-cyclopropanedicarboxylic acid and isobutyric acid were 80% and 78%, respectively.

3,3-dimethyl-1,2-cyclopropanedicarboxylic acid: ¹H NMR (500 MHz, DMSO-d₆) δ=12.41 (s, 1H), 1.89 (s, 1H), 1.19 (s, 3H). ¹³C NMR (126 MHz, DMSO-d₆) δ=171.77, 33.63, 29.55, 20.74, 20.65. HR-ESI-MS calculated for C₇H₁₀O₄[M−H]⁻: 157.15, found 157.17.

Synthesis of 3,3-dimethyl-1,2-cyclopropanedicarboxylic acid-1,2-dimethyl ester: 3,3-dimethyl-1,2-cyclopropanedicarboxylic acid (0.26 g, 0.0016 mol) was added to a 20 mL one-neck round-bottom flask, then 5 mL methanolic HCl 1M was poured into this flask. The reaction was reflux for 2 h. Once finishing reaction, 10 mL hexane was added to the flask. Two layers were divided by separating funnel. The organic layer was dried over anhydrous Na₂SO₄ and evaporated to afford desired product as a colorless oil with the yield of 79%.

3,3-dimethyl-1,2-cyclopropanedicarboxylic acid-1,2-dimethyl ester: ¹H NMR (500 MHz, Chloroform-d) δ=3.89 (d, J=15.6, 1H), 3.70 (d, J=15.9, 2H), 3.65 (s, 80H), 1.40 (s, 1H), 1.25 (s, 86H), 1.21 (s, 2H), 0.80 (s, 1H). ¹³C NMR (126 MHz, Chloroform-d) δ=170.93, 51.94, 33.58, 30.47, 20.44.

Synthesis of 1,2-cyclopropanedicarboxylic acid-3,3-dimethyl-1,2-dihydrazide: 0.6 mL 0.6 mL N₂H₄ (0.017 mol) was added slowly to 15 mL methanol contain 0.33 g 3,3-dimethyl-1,2-cyclopropanedicarboxylic acid-1,2-dimethyl ester (0.0017 mol) in 150 mL one-neck round-bottom flask. The reaction was refluxed for 6 h. After cooling down, desired product (88% in yield) is collected as a white powder after filtration and washing with methanol.

1,2-cyclopropanedicarboxylic acid-3,3-dimethyl-1,2-dihydrazide: ¹H NMR (500 MHz, DMSO-d₆) δ=9.13 (s, 1H), 4.14 (s, 2H), 1.92 (s, 1H), 1.11 (s, 3H). ¹³C NMR (126 MHz, DMSO-d₆) δ=169.48, 31.62, 27.64, 20.61. HR-ESI-MS calculated for C₇H₁₄N₄O₂[M+Na]⁺: 209.19, found 209.22.

Synthesis of cyclopropane-2,3-diisocyanato-1,1-dimethyl: The flow system was set up as described in FIG. 2.

Solution 1: sodium nitrite (0.34 g, 0.005 mol) was distilled into 50 mL deionized water.

Solution 2: pure toluene

Solution 3: 1,2-cyclopropanedicarboxylic acid-3,3-dimethyl-1,2-dihydrazide (0.46 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a colorless liquid (57%) and stored at a temperature of 4° C.

Cyclopropane-2,3-diisocyanato-1,1-dimethyl: ¹H NMR (500 MHz, Chloroform-d) δ=7.30 (t, J=7.6, 2H), 7.23-7.17 (m, 2H), 2.64 (s, 1H), 2.40 (s, 3H), 1.41-1.21 (m, 1H), 1.20 (s, 3H). ¹³C NMR (126 MHz, Chloroform-d) δ=138.01, 129.19, 128.43, 128.38, 125.46, 125.42, 122.00, 42.09, 42.02, 41.98, 24.89, 21.60, 21.56, 19.11, 19.06. HR-ESI-MS calculated for C₇H₈N₂O₂[M+MeOH+Na]⁺: 239.23, found 239.24.

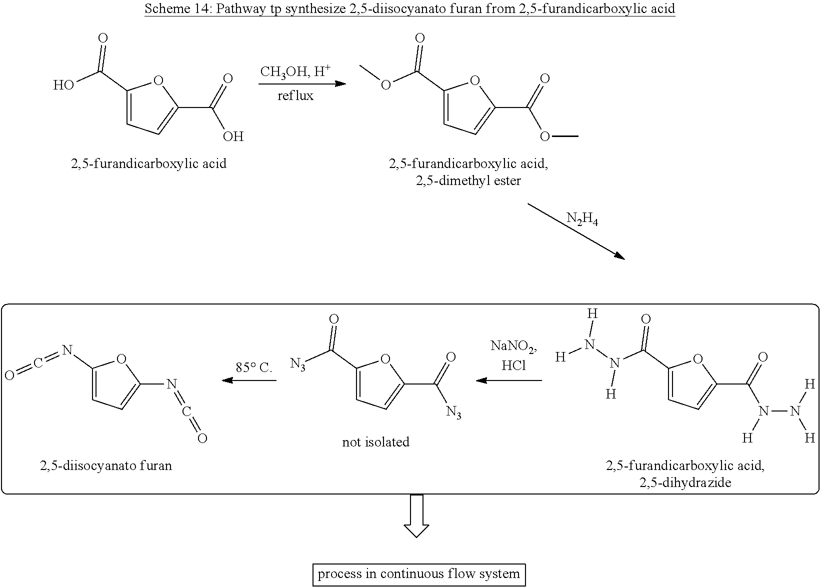

Synthesis of 2,5-furandicarboxylic acid, 2,5-dimethyl ester: 2,5-furandicarboxylic acid (1.67 g, 0.011 mol) was added to a 50 mL one-neck round-bottom flask, then 20 mL methanolic HCl 1M was poured into this flask. The reaction was reflux for 2 h. Once finishing reaction, 100 mL hexane was added to the flask. Two layers were divided by separating funnel. The organic layer was dried over anhydrous Na₂SO₄ and evaporated to afford desired product as a white powder with the yield of 59%.

2,5-furandicarboxylic acid, 2,5-dimethyl ester: ¹H NMR (500 MHz, DMSO-d₆) δ=7.42-7.34 (m, 4H), 7.31-7.24 (m, 2H), 3.82 (d, J=4.0, 12H), 3.34 (s, 2H), 2.47 (d, J=5.2, 1H). ¹³C NMR (126 MHz, DMSO-d₆) δ=159.34, 158.39, 147.94, 146.57, 146.21, 119.64, 52.94.

Synthesis of 2,5-furandicarboxylic acid, 2,5-dihydrazide: 2.5 mL N₂H₄ (0.08 mol) was added slowly to 25 mL methanol contain 1.49 g 2,5-furandicarboxylic acid, 2,5-dimethyl ester (0.008 mol) in 150 mL one-neck round-bottom flask. The reaction was refluxed for 6 h. After cooling down, desired product (74% in yield) is collected as a light pink powder after filtration and washing with methanol.

2,5-furandicarboxylic acid, 2,5-dihydrazide: ¹H NMR (500 MHz, DMSO-d₆) δ=7.07 (s, 1H), 6.98 (d, J=3.4, 1H), 6.68 (d, J=3.4, 1H), 3.34 (s, 1H). ¹³C NMR (126 MHz, DMSO-d₆) δ=157.44, 147.74, 115.13, 114.06, 113.86. HR-ESI-MS calculated for C₆H₈N₄O₃[M+H]⁺: 185.15, found 185.16.

Synthesis of 2,5-diisocyanato furan: The flow system was set up as described in FIG. 2.

Solution 1: sodium nitrite (0.34 g, 0.005 mol) was distilled into 50 mL deionized water.

Solution 2: pure toluene

Solution 3: 2,5-furandicarboxylic acid, 2,5-dihydrazide (0.46 g, 0.0025 mol) was dissolved in 50 mL deionized water and 0.8 mL aqueous HCl 6N.

All solutions were pumped with a flow rate of 1 mL min⁻¹. The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a colorless liquid (71%) and stored at a temperature of 4° C.

2,5-diisocyanato furan: ¹H NMR (500 MHz, Chloroform-d) δ=7.41-7.35 (m, 1H), 7.31-7.20 (m, 4H), 7.20-7.12 (m, 3H), 5.32 (d, J=3.9, 1H), 5.11 (s, 4H), 4.12 (q, J=7.0, 1H), 2.36 (s, 2H), 2.05 (s, 2H), 2.00 (s, 2H), 1.37-1.23 (m, 6H), 0.92-0.82 (m, 1H). ¹³C NMR (126 MHz, Chloroform-d) δ=162.39, 148.46, 137.99, 129.15, 128.34, 126.71, 125.41, 120.19, 119.35, 31.71, 22.77, 21.56, 14.24. HR-ESI-MS calculated for C₆H₂N₂O₃[M+MeOH+H]⁺: 183.13, found 183.04.

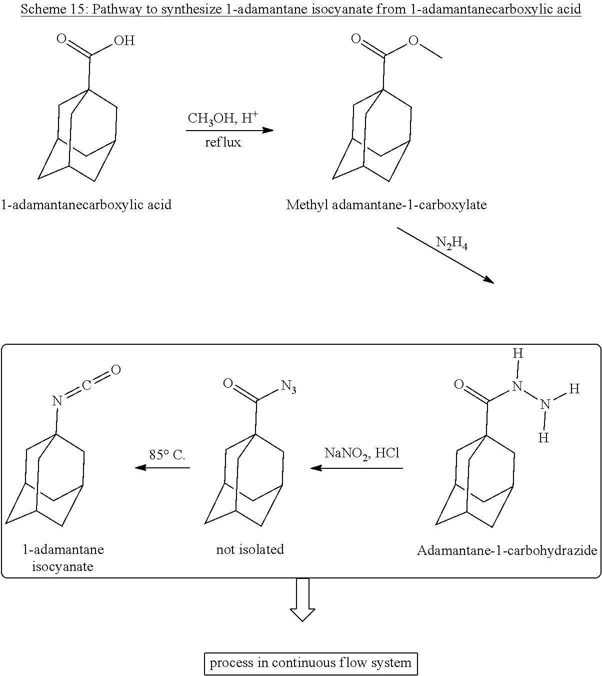

Synthesis of methyl adamantine-1-carboxylate: 1-adamantanecarboxylic acid (3.00 g, 0.016 mol) was added to a 50 mL one-neck round-bottom flask, then 20 mL methanolic HCl 1M was poured into this flask. The reaction was reflux for 2 h. Once finishing reaction, 100 mL hexane was added to the flask. Two layers were divided by separating funnel. The organic layer was dried over anhydrous Na₂SO₄ and evaporated to afford desired product as a colorless crystal with the yield of 75%.

Methyl adamantine-1-carboxylate: ¹H NMR (500 MHz, Chloroform-d) δ=3.63 (s, 1H), 1.99 (q, J=3.3, 1H), 1.87 (d, J=3.1, 2H), 1.75-1.65 (m, 2H). ¹³C NMR (126 MHz, Chloroform-d) δ=178.34, 51.64, 38.94, 36.58, 28.02.

Synthesis of Adamantane-1-carbohydrazide: 3.6 mL N₂H₄ (0.12 mol) was added slowly to 25 mL methanol contain 2.26 g methyl adamantine-1-carboxylate (0.012 mol) in 50 mL one-neck round-bottom flask. The reaction was refluxed for 6 h. After cooling down, desired product (75% in yield) is collected as a white powder after filtration and washing with methanol.

Adamantane-1-carbohydrazide: ¹H NMR (500 MHz, DMSO-d₆) δ=8.65 (s, 0H), 3.54 (s, 1H), 1.93 (p, J=3.1, 1H), 1.77 (d, J=3.0, 2H), 1.69-1.57 (m, 2H). ¹³C NMR (126 MHz, DMSO-d₆) δ=177.50, 51.91, 38.93, 36.43, 27.83. HR-ESI-MS calculated for C₁₁H₁₈N₂O [M+H]⁺: 195.27, found 195.26.

Synthesis of 1-adamantane isocyanate: The flow system was set up as described in FIG. 2.

Solution 1: sodium nitrite (0.34 g, 0.005 mol) was distilled into 50 mL deionized water.

Solution 2: pure toluene

Solution 3: Adamantane-1-carbohydrazide (0.48 g, 0.0025 mol) was dissolved in 50 mL deionized water and 0.8 mL aqueous HCl 6N.

All solutions were pumped with a flow rate of 1 mL min⁻¹, The 1.62 mL coiled reactor was cooled down to 5-10° C. in a cold bath, resulting in a residence time of 97 s. The outlet then entered the Zaiput separator, which provides continuous separation of the two immiscible liquids, toluene and water. After complete separation, water exited on one side and was collected in a waste Erlenmeyer flask while the organic phase exited on the other side. The organic phase containing the azide product was directly sent to the drying unit with anhydrous sodium sulfate to remove trace water. Finally, the outlet was injected into a second 5 mL coiled reactor, which was heated to 85° C. in a sand bath. The outlet containing desired product was placed in an Erlenmeyer flask. After removing solvent, desired product was collected as a brown powder (61%) and stored at a temperature of 4° C.

1-adamantane isocyanate: ¹H NMR (500 MHz, Chloroform-d) δ=7.59-7.52 (m, 4H), 7.48 (s, 4H), 3.92 (s, 1H), 2.65 (s, 7H), 2.33 (q, J=3.2, 1H), 2.25 (d, J=3.0, 2H), 2.09-1.98 (m, 2H). ¹³C NMR (126 MHz, Chloroform-d) δ=178.43, 138.15, 129.45, 128.65, 125.75, 51.77, 39.30, 36.93, 28.43, 21.78. HR-ESI-MS calculated for C₁₁H₁₅NO [M+MeOH]⁺: 209.31, found 209.90.

### Example 2

Flow Chemistry to Synthesize Diisocyanate from Dicarboxylic Acid Originate from Algae Oil

Azelaic acid isolated from algae biomass follows the shown procedure in FIG. 3. The obtained azelaic acid is then converted to azelaic dihydrazide using the hydrazine under acid condition (Scheme 2). The azelaic dihydrazide is stable solid and easy to store at room temperature. The azelaic dihydrazide is soluble in aqueous acid that is suitable for flow chemistry.

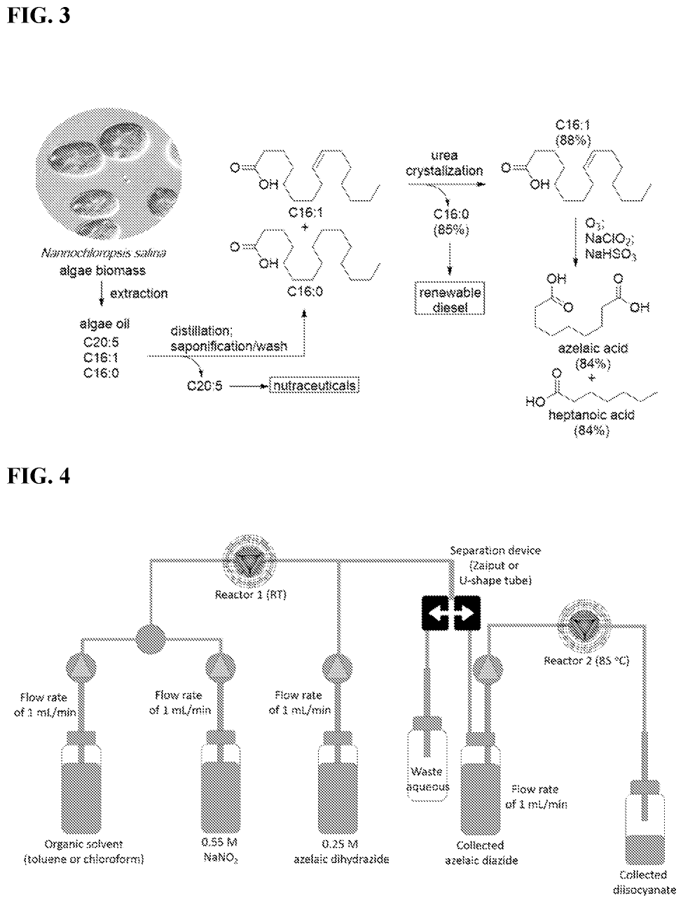

The flow system is set up as illustrated in FIG. 4. In this set up, we applied the HPLC pumps to pump reagents to the reactors. The separation device could be Zaiput separation or U-shape tube. The flow rate for each pump is adjust at 1 mL·min⁻¹. For this flow rate, this system could produce around 3.5 g·h⁻¹. The flask for collecting isocyante product should kept under inert gas such as Ar or N₂.

The flow system is applied to scale up producing hexamethylene and heptamethylene diisocyanate from dicarboxylic acid derivatives. After collecting the product, removing solvent provides isocyanate as a clear liquid. The detail structure is analyzed by both ¹H & ¹³C NMR.

The section headings used herein are for organizational purposes only and are not to be construed as limiting the subject matter described. All documents, or portions of documents, cited in the application are hereby expressly incorporated by reference in their entirety for any purpose.

While various embodiments and aspects are shown and described herein, it will be obvious to those skilled in the art that such embodiments and aspects are provided by way of example only. Numerous variations, changes, and substitutions will now occur to those skilled in the art. Various alternatives to the embodiments and aspects described herein may be used.

### REFERENCES

(1) Camaroglio, D.; Martina, K.; Palmisano, G.; Penoni, A.; Domini, C.; Cravotto, G. One-pot sequential synthesis of isocyanates and urea derivatives via a microwave-assisted Staudinger-aza-Wittig reaction. Beilstein J. Org. Chem. 2013, 9, 2378-2386.
(2) Six, C.; Richter, F. Isocyanates, Organic. Ullmann's Encyclopedia of Industrial Chemistry 2003.
(3) Knolker, H. J.; Braxmeier, T.; Schlechtingen, G. A Novel Method for the Synthesis of Isocyanates Under Mild Conditions. Angew. Chem. Int. Ed. Engl 1995, 34, 2497-2500.
(4) Kreye, O.; Mutlu, H.; Meier, M. A. R. Sustainable routes to polyurethane precursors. Green Chem. 2013, 15, 1431-1456.
(5) Tsai, J. H.; Takaoka, L. R.; Powell, N. A.; Nowick, J. S. Synthesis Of Amino Acid Ester Isocyanates. Org. Synth. 2002, 78, 220-223.
(6) Nowick, J. S.; Holmes, D. L.; Noronha, G.; Smith, E. M.; Nguyen, T. M.; Huang, S.-L. Synthesis of Peptide Isocyanates and Isothiocyanates. J. Org. Chem. 1996, 61, 3929-3934.
(7) Wegner, J.; Ceylan, S.; Kirschning, A. Ten key issues in modern flow chemistry. Chem. Commun. 2011, 47, 4583-4592.
(8) Britton, J.; Raston, C. L. Multi-step continuous-flow synthesis. Chem. Soc. Rev. 2017, 46, 1250-1271.
(9) Baumann, M.; Baxendale, I. R.; Ley, S. V.; Nikbin, N.; Smith, C. D. Azide monoliths as convenient flow reactors for efficient Curtius rearrangement reactions. Org. Biomol. Chem. 2008, 6, 1587-1593.
(10) Britton, J.; Jamison, T. F. The assembly and use of continuous flow systems for chemical synthesis. Nat. Protoc. 2017, 12, 2423-2447.
(11) Baumann, M.; Baxendale, I. R.; Ley, S. V.; Nikbin, N.; Smitha, C. D.; Tierney, J. P. A modular flow reactor for performing Curtius rearrangements as a continuous flow process. Org. Biomol. Chem. 2008, 6, 1577-1586
(12) Trojanowicz, M. Flow chemistry vs. flow analysis. Talanta 2016, 146, 621-640.
(13) Sprecher, H.; Payin, M. N. P.; Weber, M.; Yilmaz, G.; Wille, G. Acyl Azide Synthesis and Curtius Rearrangements in Microstructured Flow Chemistry Systems. J. Flow Chem. 2012, 1, 20-23.
(14) Hai, T. A. P.; Neelakantan, N.; Tessman, M.; Sherman, S. D.; Griffin, G.; Pomeroy, R.; Mayfield, S. P.; Burkart, M. D. Flexible polyurethanes, renewable fuels, and flavorings from a microalgae oil waste stream. Green Chem. 2020, 10.1039/dOgc00852d.
(15) Mata, A.; Weigl, U.; Flogel, O.; Baur, P.; Hone, C. A.; Kappe, C. O. Acyl azide generation and amide bond formation in continuous-flow for the synthesis of peptides. React. Chem. Eng. 2020, 5, 645-650.
(16) Brase, S.; Gil, C.; Knepper, K.; Zimmermann, V. Organic Azides: An Exploding Diversity of a Unique Class of Compounds. Angew. Chem. Int. Ed. 2005, 44, 5188-5240.
(17) Fortt, R.; Wootton, R. C. R.; Mello, A. J. d. Continuous-Flow Generation of Anhydrous Diazonium Species: Monolithic Microfluidic Reactors for the Chemistry of Unstable Intermediates. Org. Proc. Res. Dev. 2003, 7, 762-768.
(18) Putrescine Dihydrochloride. Org. Synth. 1956, 36, 69 DOI: 10.15227/orgsyn.15036.10069.
(19) Biochemistry, D. o. C. a. The University of North Carolina at Greensboro https://chem.uncg.edu/croatt/flow-chemistry/.
(20) Al-Amiery, A. A.; Kassim, F. A. B.; Kadhum, A. A. H.; Mohamad, A. B. Synthesis and characterization of a novel eco-friendly corrosion inhibition for mild steel in 1M hydrochloric acid. Scientific Reports 2016, 6, 19890.
(21) Poolman, J. M.; Maity, C.; Boekhoven, J.; Mee, L. v. d.; Sage, V. A. A. I.; Groenewold, G. J. M.; Kasteren, S. I. v.; Versluis, F.; Esch, J. H. v.; Eelkema, R. A toolbox for controlling the properties and functionalisation of hydrazone-based supramolecular hydrogels. J. Mater. Chem. B 2016, 4, 852-859.
(22) Rogers, L.; Jensen, K. F. Continuous manufacturing—the Green Chemistry promise?Green Chem. 2019, 21, 3481-3498.
(23) Macor, J. E.; Mullen, G.; Verhoest, P.; Sampognaro, A.; Shepardson, B.; Mack, R. A. A Chiral Synthesis of (−)-Spiro[1-azabicyclo[2.2.2]octane-3,5′-oxazolidin-2′-one]: A Conformationally Restricted Analogue of Acetylcholine That Is a Potent and Selective α7 Nicotinic Receptor Agonist. J. Org. Chem. 2004, 69, 6493-6495.
(24) Zimmermann, H.; Rudolph, J. Protonic States and the Mechanism of Acid-Catalysed Esterification. Angewandte Chemie Int. Ed. 1965, 4, 40-49.
(25) Micovic, M. Ethyl Adipate. Org. Synth. Coll. 10.15227/orgsyn.000.0001, 2, 264-265.
(26) Sonnenberger, S.; Lange, S.; Langner, A.; Neubert, R. H. H.; Dobner, B. Synthesis of ceramides NS and NP with perdeuterated and specifically Ω deuterated N-acyl residues. J. Labelled Comp. Radiopharm. 2016, 59, 531-542.
(27) Audubert, C.; Lebel, H. Mild Esterification of Carboxylic Acids via Continuous Flow Diazotization of Amines. Org. Lett. 2017, 19, 4407-4410.
(28) Hu, H.; Ota, H.; Baek, H.; Shinohara, K.; Mase, T.; Uozumi, Y.; Yamada, Y. M. A. Second-Generation meta-Phenolsulfonic Acid-Formaldehyde Resin as a Catalyst for Continuous-Flow Esterification. Org. Lett. 2020, 22, 160-163.
(29) Britton, J.; Dalziel, S. B.; Raston, C. L. The synthesis of di-carboxylate esters using continuous flow vortex fluidics. Green Chem. 2015, 18, 2193-2200.
(30) Adeyemi, A.; Bergman, J.; Branalt, J.; Savmarker, J.; Larhed, M. Continuous Flow Synthesis under High-Temperature/High-Pressure Conditions Using a Resistively Heated Flow Reactor. Org. Process Res. Dev. 2017, 21, 947-955.
(31) Zhang, X.; Breslav, M.; Grimm, J.; Guan, K.; Huang, A.; Liu, F.; Maryanoff, C. A.; Palmer, D.; Patel, M.; Qian, Y.; Shaw, C.; Sorgi, K.; Stefanick, S.; Xu, D. A New Procedure for Preparation of Carboxylic Acid Hydrazides. J. Org. Chem. 2002, 67, 9471-9474.
(32) Cougnon, F. B. L.; Caprice, K.; Pupier, M.; Bauzi, A.; Frontera, A. A Strategy to Synthesize Molecular Knots and Links Using the Hydrophobic Effect. J. Am. Chem. Soc. 2018, 140, 12442-12450.
(33) Plutschack, M. B.; Pieber, B.; Gilmore, K.; Seeberger, P. H. The Hitchhiker's Guide to Flow Chemistry. Chem. Rev. 2017, 117, 11796-11893.
(34) Nettekoven, M. Combinatorial Synthesis of 5-Aryl-[1,2,4]-triazolo-[1,5-a]-pyridine Derivatives as Potential Inhibitors of the Adenosine 2A Receptor. Synlett 2001, 12.
(35) Burgos, A.; Ellames, G. J. J. Labelled Comp. Radiopharm. 1998, XLI, 443-449.
(36) Baxendale, I. R. The integration of flow reactors into synthetic organic chemistry. J. Chem. Technol. Biotechnol. 2013, 88, 519-552.
(37) Sagandira, C. R.; Watts, P. Safe and highly efficient adaptation of potentially explosive azide chemistry involved in the synthesis of Tamiflu using continuous-flow technology. Beilstein J. Org. Chem. 2019, 15, 2577-2589.
(38) Sagandira, C. R.; Watts, P. Synthesis of Amines, Carbamates and Amides by Multi-Step Continuous Flow Synthesis. Eur. J. Org. Chem. 2017, 6554-6565.
(39) Dhanya, R. P.; Herath, A.; Sheffler, D. J.; Cosford, N. D. P. A combination of flow and batch mode processes for the efficient preparation of mGlu2/3 receptor negative allosteric modulators (NAMs). Tetrahedron 2018, 74, 3165-3170.
(40) Herath, A.; Cosford, N. D. P. Continuous-flow synthesis of highly functionalized imidazo-oxadiazoles facilitated by microfluidic extraction. Beilstein J. Org. Chem. 2017, 13, 239-246.
(41) Stedman, G.; Uysal, N. Kinetics and Mechanism of the Reaction between Nitrous Acid and Arylhydrazines. J. Chem. Soc., Perkin Trans. 1977, 2, 667-671.
(42) Bemheim, P.; Dobos, A.; Doherty, A. M. M.; Haine, N.; Stedman, G. Kinetics and mechanism of the reaction of nitrous acid with 2,4-dinitrophenylhydrazine. J. Chem. Soc., Perkin Trans. 1996, 2, 275-280.
(43) Ghosh, A. K.; Sarkar, A.; Brindisi, M. The Curtius Rearrangement: Mechanistic Insight and Recent Applications in Natural Product Syntheses. Org Biomol Chem. 2018, 16, 2006-2027.
(44) Newman, M. S.; Jr., S. H. L.; Garrett, A. B. Solvent Effect in the Curtius Rearrangement of Benzazide. J. Am. Chem. Soc. 1947, 69, 113-116.
(45) Kima, J.-G.; Jang, D. O. Direct Synthesis of Acyl Azides from Carboxylic Acids by the Combination of Trichloroacetonitrile, Triphenylphosphine and Sodium Azide. Synlett 2008, 13, 2072-2074.
(46) Brandt, J. C.; Wirth, T. Controlling hazardous chemicals in microreactors: Synthesis with iodine azide. Beilstein J. Org. Chem. 2009, 5, 1-6.
(47) Hojabri, L.; Kong, X.; Narine, S. S. Fatty Acid-Derived Diisocyanate and Biobased Polyurethane Produced from Vegetable Oil: Synthesis, Polymerization, and Characterization. Biomacromolecules 2009, 10, 884-891.
(48) More, A. S.; Lebarbe, T.; Maisonneuve, L.; Gadenne, B.; Alfos, C.; Cramail, H. Novel fatty acid based di-isocyanates towards the synthesis of thermoplastic polyurethanes. Eur. Polym. J. 2013, 49, 823-833.
(49) Ende, D. J. a.; DeVries, K. M.; Clifford, P. J.; Brenek, S. J. A Calorimetric Investigation To Safely Scale-Up a Curtius Rearrangement of Acryloyl Azide. Org. Proc. Res. Dev. 1998, 6, 382-392.
(50) Zhao, J.; Gimi, R.; Katti, S.; Reardon, M.; Nivorozhkin, V.; Konowicz, P.; Lee, E.; Sole, L.; Green, J.; Siegel, C. S. Process Development of a GCS Inhibitor Including Demonstration of Lossen Rearrangement on Kilogram Scale. Org. Process Res. Dev. 2020, 10.1021/op500379a.
(51) Papeo, G.; Posteri, H.; Vianello, P.; Varasi, M. Nicotinoyl Azide (NCA)-Mediated Mitsunobu Reaction: An Expedient One-Pot Transformation of Alcohols into Azides. Synthesis 2004, 17, 2886-2892.
(52) Smith, P. A. S. The Curtius Reaction. Organic Reactions 2011, 337-449.
(53) Ghosh, A. K.; Brindisi, M.; Sarkar, A. The Curtius Rearrangement: Applications in Modern Drug Discovery and Medicinal Chemistry. ChemMedChem 2018, 13, 2351-2373.
(54) Lebel, H.; Leogane, O. Curtius Rearrangement of Aromatic Carboxylic Acids to Access Protected Anilines and Aromatic Ureas. Org. Lett. 2006, 25, 5717-5720.
(55) Hojabri, L.; Kong, X.; Narine, S. S. Novel Long Chain Unsaturated Diisocyanate from Fatty Acid: Synthesis, Characterization, and Application in Bio-Based Polyurethane. J. Polym. Sci. Part A 2010, 48, 3302-3310
(56) Koch, T. A.; Krause, K. R.; Mehdizadeh, M. Improved safety through distributed manufacturing of hazardous chemicals. Process Saf. Prog. 1997, 16, 23-24.

What is claimed is:

1. A continuous flow process for producing an isocyanate, the process comprising:
(i) mixing an acyl hydrazide with an aqueous solution comprising nitrous acid in flow to form a first solution comprising an acyl azide and water;
(ii) mixing an organic solvent with the first solution in flow to produce a second solution comprising the acyl azide compound, the organic solvent, and water;
(iii) removing water from the second solution in flow to produce a third solution comprising the azide compound and the organic solvent; and
(iv) heating the third solution comprising the azide compound in flow to produce the isocyanate.

2. The process of claim 1, further comprising drying the third solution in flow prior to heating the third solution.

3. The process of claim 1, wherein the organic solvent comprises chloroform, toluene, benzene, methyl tert-butyl ether, tetrahydrofuran, or a mixture of two or more thereof.

4. The process of claim 1, wherein the ratio of acyl hydrazide to nitrous acid is from about 1:1 to about 1:3.

5. The process of claim 4, wherein the ratio of acyl hydrazide to nitrous acid is from about 1:2.

6. The process of claim 1, wherein the volume of organic solvent to the volume of the first solution is from about 2:1 to about 1:2.

7. The process of claim 6, wherein the volume of organic solvent to the volume of the first solution is from about 1:1.

8. The process of any claim 1, comprising mixing the acyl hydrazide with the aqueous solution in flow at a temperature from about 0° C. to about 30° C.

9. The process of claim 8, comprising mixing the acyl hydrazide with the aqueous solution in flow at a temperature from about 20° C. to about 25° C.

10. The process of claim 1, comprising heating the third solution in flow at a temperature from about 65° C. to about 120° C.

11. The process of claim 10, comprising heating the third solution in flow at a temperature from about 85° C. to about 100° C.

12. The process of claim 1, wherein the isocyanate is a diisocyanate.

13. The process of claim 12, wherein the acyl hydrazide has the structure: 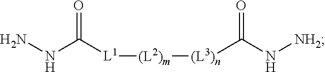
wherein the acyl azide has the structure: 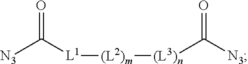
wherein the isocyanate has the structure:
O═C═N-L¹-(L²)ₘ-(L³)ₙ-N═C═O;
wherein L¹, L², and L³ are each independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
m is 0 or 1; and
n is 0 or 1.

14. The process of claim 13, wherein L¹, L², and L³ are each independently substituted or unsubstituted C₁-C₁₂alkylene, substituted or unsubstituted C₅-C₆cycloalkylene, substituted or unsubstituted C₅-C₆ arylene, or substituted or unsubstituted 5 or 6 membered heteroarylene.

15. The process of claim 13, wherein m and n are 0.

16. The process of claim 13, wherein m and n are 1.

17. The process of claim 13, wherein m is 1 and n is 0.

18. The process of claim 12, wherein the diisocyanate is methylene diphenyl diisocyanate.

19. The process of claim 12, wherein the diisocyanate is toluene diisocyanate.

20. The process of claim 12, wherein the diisocyanate is hexamethylene diisocyanate.

21. The process of claim 12, wherein the diisocyanate is isophorone diisocyanate.

22. The process of claim 12, wherein the diisocyanate is tetramethylxylene diisocyanate.

23. The process of claim 12, wherein the diisocyanate is: OCN—CH₂—NCO; OCN—(CH₂)₅—NCO; OCN—(CH₂)₇—NCO;

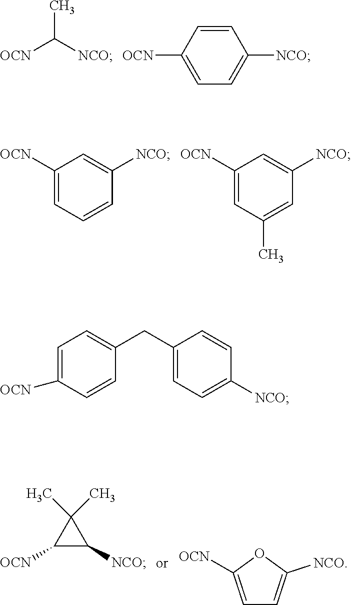

24. The process of claim 1, wherein the isocyanate is a monoisocyanate.

25. The process of claim 24, wherein the acyl hydrazide has the structure: 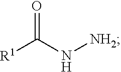
wherein the acyl azide has the structure: 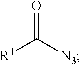
wherein the isocyanate has the structure:
R¹—N═C═O;
wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

26. The process of claim 25, wherein R¹ is substituted or unsubstituted C₂-C₂₀alkyl, substituted or unsubstituted cycloalkyl, substituted or unsubstituted C₅-C₆ aryl, or substituted or unsubstituted 5 or 6 membered heteroaryl.

27. The process of claim 24, wherein the monoisocyanate is: CH₃(CH₂)₅NCO; CH₃(CH₂)₁₄NCO;

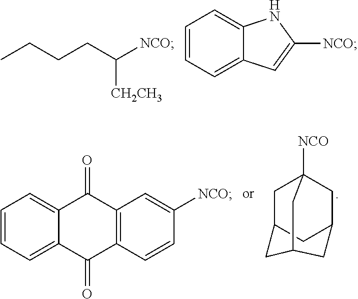

28. A process for producing at least one gram of an isocyanate, the process comprising:
(i) mixing an acyl hydrazide with an aqueous solution comprising nitrous acid to form a first solution comprising an acyl azide and water;
(ii) mixing an organic solvent with the first solution to produce a second solution comprising the azide compound, the organic solvent, and water;
(iii) removing water from the second solution to produce a third solution comprising the azide compound and the organic solvent; and
(iv) heating the third solution comprising the azide compound to produce at least one gram of the isocyanate.

29. The process of claim 28 for producing at least one kilogram of an isocyanate.

30. The process of claim 29 for producing at least 500 kilograms of an isocyanate.

31. The process of any claim 28, further comprising drying the third solution in flow prior to heating the third solution.

32. The process of claim 28, wherein the organic solvent comprises chloroform, toluene, benzene, methyl tert-butyl ether, tetrahydrofuran, or a mixture of two or more thereof.

33. The process of claim 28, wherein the ratio of acyl hydrazide to nitrous acid is from about 1:1 to about 1:3.

34. The process of claim 33, wherein the ratio of acyl hydrazide to nitrous acid is from about 1:2.

35. The process of claim 28, wherein the volume of organic solvent to the volume of the first solution is from about 2:1 to about 1:2.

36. The process of claim 35, wherein the volume of organic solvent to the volume of the first solution is from about 1:1.

37. The process of claim 28, comprising mixing the acyl hydrazide with the aqueous solution in flow at a temperature from about 0° C. to about 30° C.

38. The process of claim 37, comprising mixing the acyl hydrazide with the aqueous solution in flow at a temperature from about 20° C. to about 25° C.

39. The process of claim 28, comprising heating the third solution in flow at a temperature from about 65° C. to about 120° C.

40. The process of claim 39, comprising heating the third solution in flow at a temperature from about 85° C. to about 100° C.

41. The process of any one of claims 28 to 40, wherein the isocyanate is a diisocyanate.

42. The process of claim 41, wherein the acyl hydrazide has the structure: 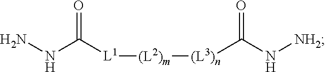
wherein the acyl azide has the structure: 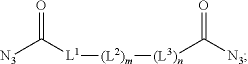
wherein the isocyanate has the structure:
O═C═N-L¹-(L²)ₘ-(L³)ₙ-N═C═O;
wherein L¹, L², and L³ are each independently substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
m is 0 or 1; and
n is 0 or 1.

43. The process of claim 42, wherein L¹, L², and L³ are each independently substituted or unsubstituted C₁-C₁₂alkylene, substituted or unsubstituted C₅-C₆cycloalkylene, substituted or unsubstituted C₅-C₆ arylene, or substituted or unsubstituted 5 or 6 membered heteroarylene.

44. The process of claim 41, wherein m and n are 0.

45. The process of claim 41, wherein m and n are 1.

46. The process of claim 41, wherein m is 1 and n is 0.

47. The process of claim 41, wherein the diisocyanate is methylene diphenyl diisocyanate.

48. The process of claim 41, wherein the diisocyanate is toluene diisocyanate.

49. The process of claim 41, wherein the diisocyanate is hexamethylene diisocyanate.

50. The process of claim 41, wherein the diisocyanate is isophorone diisocyanate.

51. The process of claim 41, wherein the diisocyanate is tetramethylxylene diisocyanate.

52. The process of claim 41, wherein the diisocyanate is: OCN—CH₂—NCO; OCN—(CH₂)₅—NCO; OCN—(CH₂)₇—NCO;

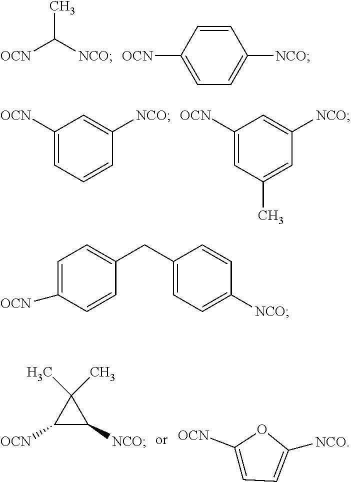

53. The process of claim 28, wherein the isocyanate is a monoisocyanate.

54. The process of claim 53, wherein the acyl hydrazide has the structure: 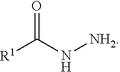
wherein the acyl azide has the structure: 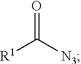
and
wherein the isocyanate has the structure:
R¹—N═C═O;
wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

55. The process of claim 54, wherein R¹ is substituted or unsubstituted C₂-C₂₀alkyl, substituted or unsubstituted cycloalkyl, substituted or unsubstituted C₅-C₆ aryl, or substituted or unsubstituted 5 or 6 membered heteroaryl.

56. The process of claim 53, wherein the monoisocyanate is: CH₃(CH₂)₅NCO; CH₃(CH₂)₁₄NCO; [chemical structure]
